(19) United States
(12) Patent Application Publication
(10) Pub. No.: US 2017/0165161 A1
(43) Pub. Date: Jun. 15, 2017

(54) OXIDIZING AGENT FOR THE OXIDATIVE COLORING AND BLONDING TREATMENT OF KERATIN FIBERS HAVING A REDUCED DAMAGING EFFECT

(71) Applicant: Henkel AG & Co. KGaA, Duesseldorf (DE)

(72) Inventors: Hartmut Manneck, Barnitz (DE); Thomas Hippe, Appen (DE); Stefan Hoepfner, Hamburg (DE); Jessica Brender, Hamburg (DE)

(73) Assignee: Henkel AG & Co. KGaA, Duesseldorf (DE)

(21) Appl. No.: 15/377,779

(22) Filed: Dec. 13, 2016

Related U.S. Application Data
(60) Provisional application No. 62/353,956, filed on Jun. 23, 2016.

(30) Foreign Application Priority Data
Dec. 14, 2015 (DE) 10 2015 225 137.3

(51) Int. Cl. A61K 8/44; A61K 8/362; A61K 8/42; A61Q 5/10; A61K 8/02; A61K 8/46; A61K 8/22; A61K 8/43

(52) U.S. Cl. CPC A61K 8/44; A61K 8/22; A61K 8/362; A61K 8/43; A61Q 5/10; A61K 8/0216; A61K 8/466; A61K 8/42; A61K 2800/882; A61K 2800/4324

(57) ABSTRACT

An oxidizing composition for oxidatively coloring or blonding hair is gentle on the hair. A method for oxidatively coloring or blonding hair protects keratin fibers from oxidative influences.

### FIELD OF THE INVENTION

The present invention generally relates to an oxidizing agent for oxidatively coloring or blonding hair that is gentle on the hair, and to a gentle method for oxidatively coloring or blonding hair, in which keratin fibers are protected from oxidative influences and/or oxidative damage to the hair is repaired.

### BACKGROUND OF THE INVENTION

The problem with oxidatively coloring or blonding hair is that the keratin fibers may be damaged by the aggressive substances. In particular, the natural hydrophobicity of the keratin fiber is reduced since the coloring or lightening agents initially have to render the hair penetrable so as to develop their effect. The water-repelling action, however, provides natural protection of the hair, and additionally is closely associated with parameters desired by the consumer, such as shine, suppleness, feel, and “laying” of the hair.

So as to overcome the aforementioned drawbacks, what are known as pre-treatment agents are available on the market, which are to protect the hair from aggressive influence. However, these often weigh the hair down or impair the success of the subsequent lightening process or coloration of the hair, and in particular the washing fastness of the color may be adversely affected by the pre-treatment agent. In addition, numerous post-treatment agents are known, which are used to attempt to repair damage caused to the hair during the oxidative coloring treatment. All of these methods, however, require a multi-stage application process, involving an application of a further hair treatment agent prior to or after coloring. This is frequently perceived as inconvenient by the consumer, since the oxidative coloring treatment itself already involves multiple work steps and an exposure time of as much as 60 minutes, making the process very complex.

The use of dicarboxylic acids such as maleic acid or succinic acid in hair care is state of the art. These are used widely in shampoos, and in particular in conditioners, so as to develop nourishing effects there. The patent application WO 2005/115314 A1, for example, discloses a method for restructuring keratin fibers, in which the keratin fibers are brought in contact with cystine and at least one dicarboxylic acid having 2 to 10 carbon atoms, wherein preferred dicarboxylic acids are selected from oxalic acid, malonic acid, succinic acid, glutaric acid, adipic acid, pimelic acid, azelaic acid, maleic acid, fumaric acid, and sorbic acid, and succinic acid is particularly preferred. The patent application DE 10051774 A1 describes the use of short-chain carboxylic acids having a molecular weight below 750 g/mol in cosmetic agents as an active ingredient for restructuring keratin fibers. The patent application EP 1174112 A discloses hair treatment agents that, in addition to an organic acid, comprise an organic solvent, a cationic surfactant, and a higher alcohol as further essential components, and that are used to repair pores formed inside hair.

Entry numbers 529647, 2061070, 743114, 1431193 and 406342 in the Mintel database disclose oxidative hair coloring or blonding agent kits that include an aqueous hydrogen peroxide preparation that includes maleic acid. None of these hydrogen peroxide preparations includes an amino acid.

It is therefore desirable to provide an agent and a method for oxidatively coloring hair using a treatment that protects hair, which overcomes the aforementioned drawbacks, without negatively influencing the color result of the oxidative coloring treatment. In particular, a coloring agent and a method should be provided, in which the hair is not weighed down and which causes preferably little damage to the hair. Furthermore, the achieved protection of the hair should command as little time as possible and preferably take place directly together with the coloring step.

Other desirable features and characteristics of the present invention will become apparent from the subsequent detailed description of the invention and the appended claims, taken in conjunction with this background of the invention.

### BRIEF SUMMARY OF THE INVENTION

An oxidative treatment agent for keratin fibers, in particular for human hair, comprising: at least one dicarboxylic acid having 2 to 10 carbon atoms and/or at least one salt of this acid/these acids; at least one amino acid; hydrogen peroxide, preferably in an amount of 0.5 to 13 wt. %, more preferably 1 to 7 wt. %, particularly preferably 2 to 6 wt. %, and especially particularly preferably 3 to 4.5 wt. % (calculated as 100% H₂O₂), in each case based on the total weight of the oxidative treatment agent according to the invention; and water, preferably in an amount of 20 to 95 wt. %, preferably 30 to 90 wt. %, particularly preferably 40 to 85 wt. %, and exceptionally preferably 50 to 80 wt. %, in each case based on the total weight of the oxidative treatment agent according to the invention, wherein the oxidative treatment agent has a pH value in the range of 2.5 to 6.5, preferably in the range of 3 to 5.5, and particularly preferably in the range of 3.5 to 5.0, each measured at 20° C.

A method for oxidatively coloring and/or lightening keratin fibers, and in particular human hair, comprising the following method steps:
I. providing an oxidative treatment agent (A), comprising at least one dicarboxylic acid having 2 to 10 carbon atoms and/or at least one salt of this acid/these acids, which is preferably selected from maleic acid, fumaric acid, succinic acid, oxalic acid, malonic acid, adipic acid, pimelic acid, suberic acid, azelaic acid, sebacic acid, and mixtures of these acids, particularly preferably selected from succinic acid, maleic acid and fumaric acid, and exceptionally preferably selected from succinic acid; furthermore at least one amino acid, which is preferably selected from amino acids of formula (VI)

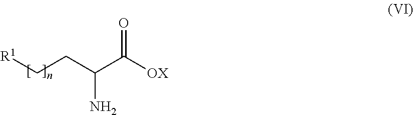

where X denotes a hydrogen atom or a monovalent or divalent cation; n denotes zero, 1, 2 or 3; R¹ denotes a group selected from an amino group, a guanidine group, a (1H-imidazole-4-yl) group, a carboxylic acid amid group —CONH₂, a 1H-indole-3-yl group, a thiol group —SH, and a methylsulfanyl group —SCH₃, or at least one salt of these amino acids; hydrogen peroxide; water; wherein the oxidative treatment agent has a pH value in the range of 2.5 to 6.5, preferably in the range of 3 to 5.5, and particularly preferably in the range of 3.5 to 5.0, each measured at 20° C.; optionally furthermore at least one substance selected from compounds of the general formula (III),

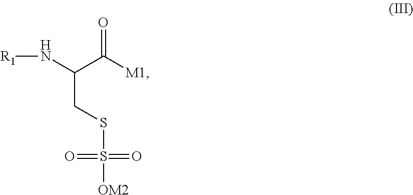

wherein R1 denotes a hydrogen atom or a structural element of formula (IV)

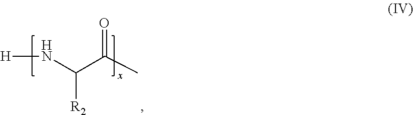

wherein a denotes an integer from 1 to 100; the group R2 in each of the structural elements of formula (IV) can each be selected independently of the preceding structural element of formula (IV); R2 denotes a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazole-4-ylmethyl group, a 1H-indole-3-ylmethyl group or a (sulfosulfanyl)methyl group; M1 denotes the group —OM2 or a structural element of formula (V),

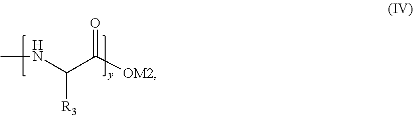

wherein y denotes an integer from 1 to 100; the group R3 in each of the structural elements of formula (V) can each be selected independently of the preceding structural element of formula (V); R3 denotes a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazole-4-ylmethyl group, a 1H-indole-3-ylmethyl group or a (sulfosulfanyl)methyl group; M2 denotes a hydrogen atom, an equivalent of a monovalent or polyvalent cation, or an ammonium ion (NH₄)⁺; at least one chelating agent, preferably in a total amount of 0.01 to 2.5 wt. %, particularly preferably in a total amount of 0.1 to 1.5 wt. %, and exceptionally preferably in a total amount of 0.2 to 0.6 wt. %, in each case based on the weight of the oxidative treatment agent (A); and at least one fatty acid amide of formula (AMID-I), preferably in a total amount of 0.1 to 10 wt. %, particularly preferably 0.2 to 5 wt. %, and exceptionally preferably 0.4 to 2 wt. %, in each case based on the weight of the oxidative treatment agent (A),

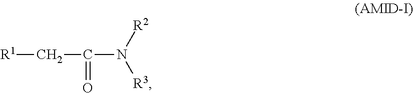

where R¹ is selected from linear and branched C4 to C22 alkyl groups, which are saturated or unsaturated, wherein the alkyl and alkenyl groups are unsubstituted or may be substituted with oxygen atoms, nitrogen atoms, hydroxyl groups, ether groups, oxyalkylene groups, polyoxyalkylene groups, carboxylic acid groups, amine groups, amide groups, halogen-containing groups, ester groups, siloxane groups, or polysiloxane groups; and alkoxylated alkyl groups of formulas:

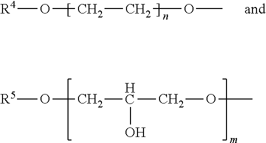

where: R⁴ and R⁵, which can be the same or different, are selected from linear or branched C4 to C22 alkyl groups, which are saturated or unsaturated; n is an integer in the range from 1 to 10, and preferably in the range from 1 to 4; and m is an integer in the range from 1 to 6; and R² and R³, which may be the same or different, are each independently of one another selected from H; linear or branched C1 to C22 alkyl groups, which are saturated or unsaturated, wherein the alkyl and alkenyl groups are unsubstituted or substituted with oxygen atoms, nitrogen atoms, hydroxyl groups, ether groups, oxyalkylene groups, and in particular oxyethylene groups, polyoxyalkylene groups, and in particular polyoxyethylene groups, carboxylic acid groups, amine groups, alkylamine groups, and in particular C1 to C3 alkylamine groups, dialkylamine groups, and in particular di-(C1-C3)-alkylamine groups, substituted or non-substituted 4,5-dihydroimidazole groups, amide groups, halogen-containing groups, ester groups, siloxane groups, or polysiloxane groups;
II. providing a composition (B), comprising at least one alkalizing agent, selected from ammonium hydroxide, monoethanolamine and sodium silicates, and mixtures thereof; optionally water; and optionally at least one oxidation dye precursor and/or at least one direct dye, wherein composition (B) preferably has a pH value in the range of 6.5 to 12.0, preferably 8 to 11.5, and particularly preferably 8.5 to 11.0, each measured at 20° C.;
III. mixing compositions (A) and (B) with each other, wherein the weight ratio of (A) to (B) is preferably in the range of 10:1 to 1:4, preferably in the range of 5:1 to 1:3, particularly preferably in the range of 4:1 to 1:2, and exceptionally preferably in the range of 2:1 to 1:1; directly thereafter
IV. applying the mixture of (A) and (B) to the keratin fibers, and in particular to the human hair; and
V. rinsing after an exposure time of 0.1 to 60 minutes, preferably 1 to 50 minutes, particularly preferably 10 to 45 minutes, and exceptionally preferably 30 to 45 minutes, and
VI. optionally further heat treatments, such as styling, conditioning and/or drying.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It was now found that oxidative treatment agents that, in addition to typical components such as hydrogen peroxide and water, comprise at least one dicarboxylic acid having 2 to 10 carbon atoms and at least one amino acid, preferably at least one amino acid of formula (VI), result in considerably improved protection of the hair during the oxidative treatment of hair, without impairing the results of the oxidative coloring or blonding treatment. Surprisingly, it was found that the presence of at least one dicarboxylic acid having 2 to 10 carbon atoms, in combination with at least one amino acid, and preferably at least one amino acid of formula (VI), protects the hair during coloring and/or lighting from damage by the high pH of the agent and by the oxidizing agent. This was able to be established, among other things, in that less hair breakage occurred during subsequent combing, and the hair lost less elasticity, as demonstrated by stress-strain measurements, than after the application of coloring and blonding agents not according to the invention.

In a first embodiment, the subject matter of the present invention is an oxidative treatment agent for keratin fibers, and in particular for human air, comprising:
a) at least one dicarboxylic acid having 2 to 10 carbon atoms and/or at least one salt of this acid/these acids,
b) at least one amino acid,
c) furthermore, hydrogen peroxide, and
d) water,
   wherein the oxidative treatment agent has a pH value in the range of 2.5 to 6.5, preferably in the range of 3 to 5.5, and particularly preferably in the range of 3.5 to 5.0, each measured at 20° C.

A further subject matter of the present invention is a method for oxidatively coloring and/or lightening keratin fibers, and in particular human hair, in which an oxidative treatment agent is applied to the keratin fibers, and in particular to the human hair, and optionally after an exposure time of 0.1 to 60 minutes, preferably 1 to 50 minutes, particularly preferably 10 to 45 minutes, and exceptionally preferably 30 to 45 minutes, is rinsed off again, wherein this treatment agent comprises
a) at least one dicarboxylic acid having 2 to 10 carbon atoms and/or at least one salt of this acid/these acids,
b) at least one amino acid,
c) hydrogen peroxide, and
d) Water
   wherein the oxidative treatment agent has a pH value in the range of 2.5 to 6.5, preferably in the range of 3 to 5.5, and particularly preferably in the range of 3.5 to 5.0, each measured at 20° C.

A further subject matter of the present invention is a method for oxidatively coloring and/or lightening keratin fibers, and in particular human hair, comprising the following method steps:
I. providing an oxidative treatment agent (A), comprising
   a) at least one dicarboxylic acid having 2 to 10 carbon atoms and/or at least one salt of this acid/these acids, which is preferably selected from maleic acid, fumaric acid, succinic acid, malic acid, oxalic acid, malonic acid, adipic acid, pimelic acid, suberic acid, azelaic acid, sebacic acid, D-tartaric acid, L-tartaric acid, mesotartaric acid, racemic tartaric acid, alpha-ketoglutaric acid, beta-ketoglutaric acid and mixtures of these acids, particularly preferably selected from succinic acid, malic acid, maleic acid and fumaric acid, and exceptionally preferably selected from succinic acid, furthermore
   b) at least one amino acid, which is preferably selected from amino acids of formula (VI)

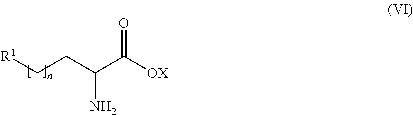

where
X denotes a hydrogen atom or a monovalent or divalent cation;
n denotes zero, 1, 2 or 3;
R¹ denotes a group selected from an amino group, a guanidine group, a (1H-imidazole-4-yl) group, a carboxylic acid amid group —CONH₂, a 1H-indole-3-yl group, a thiol group —SH, and a methylsulfanyl group —SCH₃, or at least one salt of these amino acids,
c) hydrogen peroxide, and
d) water,
e) wherein the oxidative treatment agent has a pH value in the range of 2.5 to 6.5, preferably in the range of 3 to 5.5, and particularly preferably in the range of 3.5 to 5.0, each measured at 20° C.,
f) optionally furthermore at least one substance selected from
   compounds of the general formula (III),

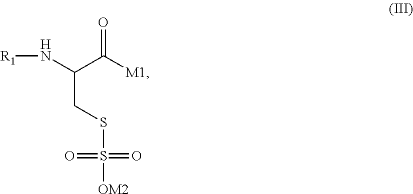

wherein
R1 denotes a hydrogen atom or a structural element of formula (IV)

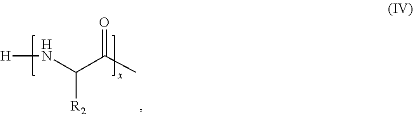

wherein
a denotes an integer from 1 to 100,
the group R2 in each of the structural elements of formula (IV) can each be selected independently of the preceding structural element of formula (IV),
R2 denotes a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazole-4-ylmethyl group, a 1H-indole-3-ylmethyl group or a (sulfosulfanyl)methyl group,
M1 denotes the group —OM2 or a structural element of formula (V),

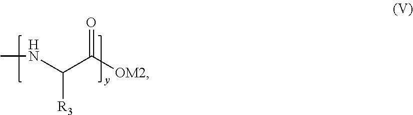

wherein
y denotes an integer from 1 to 100,
the group R3 in each of the structural elements of formula (V) can each be selected independently of the preceding structural element of formula (V),
R3 denotes a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazole-4-ylmethyl group, a 1H-indole-3-ylmethyl group or a (sulfosulfanyl)methyl group,
M2 denotes a hydrogen atom, an equivalent of a monovalent or polyvalent cation, or an ammonium ion (NH₄)⁺, and
at least one chelating agent, preferably in a total amount of 0.01 to 2.5 wt. %, particularly preferably in a total amount of 0.1 to 1.5 wt. %, and exceptionally preferably in a total amount of 0.2 to 0.6 wt. %, in each case based on the weight of the oxidative treatment agent (A), and
at least one fatty acid amide of formula (AMID-I), preferably in a total amount of 0.1 to 10 wt. %, particularly preferably 0.2 to 5 wt. %, and exceptionally preferably 0.4 to 2 wt. %, in each case based on the weight of the oxidative treatment agent (A)

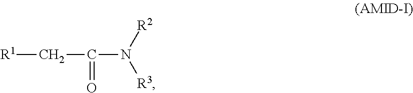

where
R¹ is selected from linear and branched C4 to C22 alkyl groups, which are saturated or unsaturated, wherein the alkyl and alkenyl groups are unsubstituted or may be substituted with oxygen atoms, nitrogen atoms, hydroxyl groups, ether groups, oxyalkylene groups, polyoxyalkylene groups, carboxylic acid groups, amine groups, amide groups, halogen-containing groups, ester groups, siloxane groups, or polysiloxane groups,
and alkoxylated alkyl groups of formulas:

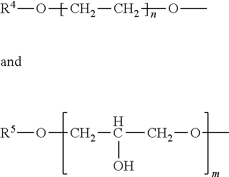

where:
R⁴ and R⁵, which can be the same or different, are selected from linear or branched C4 to C22 alkyl groups, which are saturated or unsaturated,
n is an integer in the range from 1 to 10, and preferably in the range from 1 to 4; and
m is an integer in the range from 1 to 6; and
R² and R³, which may be the same or different, are each independently of one another selected from H; linear or branched C1 to C22 alkyl groups, which are saturated or unsaturated, wherein the alkyl and alkenyl groups are unsubstituted or substituted with oxygen atoms, nitrogen atoms, hydroxyl groups, ether groups, oxyalkylene groups, and in particular oxyethylene groups, polyoxyalkylene groups, and in particular polyoxyethylene groups, carboxylic acid groups, amine groups, alkylamine groups, and in particular C1 to C3 alkylamine groups, dialkylamine groups, and in particular di-(C1-C3)-alkylamine groups, substituted or non-substituted 4,5-dihydroimidazole groups, amide groups, halogen-containing groups, ester groups, siloxane groups, or polysiloxane groups,
II. providing a composition (B), comprising
   g) at least one alkalizing agent, selected from ammonium hydroxide, monoethanolamine and sodium silicates, and mixtures thereof,
   h) optionally water, and
   i) optionally at least one oxidation dye precursor and/or at least one direct dye,
III. mixing compositions (A) and (B) with each other, wherein the weight ratio of (A) to (B) is preferably in the range of 10:1 to 1:4, preferably in the range of 5:1 to 1:3, particularly preferably in the range of 4:1 to 1:2, and exceptionally preferably in the range of 2:1 to 1:1, directly thereafter
IV. applying the mixture of (A) and (B) to the keratin fibers, and in particular to the human hair, and
V. rinsing after an exposure time of 0.1 to 60 minutes, preferably 1 to 50 minutes, particularly preferably 10 to 45 minutes, and exceptionally preferably 30 to 45 minutes, and
VI. optionally further heat treatments, such as styling, conditioning and/or drying.

Keratin fibers, keratin-containing fibers or keratinic fibers shall preferably be understood to mean human hair, furthermore furs, wool and feathers. According to the invention, “directly thereafter” shall be understood to mean a time period of 1 to 600 seconds.

Dicarboxylic Acids Having 2 to 10 Carbon Atoms and/or at Least One Salt of this Acid/these Acids

Preferred dicarboxylic acids according to the invention having 2 to 10 carbon atoms are selected from maleic acid, fumaric acid, malic acid, succinic acid, oxalic acid, malonic acid, adipic acid, pimelic acid, suberic acid, azelaic acid, sebacic acid, D-tartaric acid, L-tartaric acid, mesotartaric acid, racemic tartaric acid, alpha-ketoglutaric acid, beta-ketoglutaric acid and mixtures of these acids. According to the invention, maleic acid, fumaric acid, malic acid and succinic acid, and mixtures of these acids are particularly preferred. According to the invention, succinic acid is exceptionally preferred. The aforementioned dicarboxylic acids considerably help reduce damage caused to the hair by the treatment agents according to the invention.

Depending on the pH value of the oxidative treatment agent according to the invention, or of composition (B) used in one of the coloring or blonding methods according to the invention, the at least one dicarboxylic acid having 2 to 10 carbon atoms may be present in the form of an undissociated acid, in partially dissociated form or in completely dissociated form. If the at least one dicarboxylic acid having 2 to 10 carbon atoms is present in partially dissociated form or in completely dissociated form, the counterion is selected from physiologically compatible cations, such as in particular the alkali metal, alkaline earth metal and zinc ions, as well as ammonium ions, alkyl ammonium ions, alkanol ammonium ions and glucammonium ions, and in particular the mono-, di- and trimethyl-, -ethyl- and -hydroxyethyl ammonium ions. Likewise preferred are the salts of the dicarboxylic acids having 2 to 10 carbon atoms with amino-C1-C6alkanols, and in particular with monoethanolamine, and amino-C1-C6-alkane diols, and in particular with 2-amino-2-methylpropane-1-ol, 2-amino-2-methylpropane-1,3-diol, 2-aminopropane-1-ol, 3-aminopropane-1-ol, 1-aminopropane-2-ol (MIPA) and 2-amino-2-(hydroxymethyl)propane-1,3-diol (TRIS), wherein the salts with monoethanolamine, 2-amino-2-methylpropane-1-ol and 2-amino-2-methylpropane-1,3-diol are particularly preferred.

Sodium, potassium, magnesium, ammonium and monoethanol ammonium ions are exceptionally preferred counterions for the partially or completely dissociated dicarboxylic acids having 2 to 10 carbon atoms. In addition, however, it is also possible to use dicarboxylic acids having 2 to 10 carbon atoms that are neutralized with alkaline reacting amino acids, such as arginine, lysine, ornithine and histidine.

The sodium, potassium, ammonium, monoethanol ammonium, lysine and arginine salts and the mixtures thereof are preferred salts of the dicarboxylic acids having 2 to 10 carbon atoms. Preferred treatment agents according to the invention contain the at least one dicarboxylic acid having 2 to 10 carbon atoms, or one or more salts thereof, in a total amount of 0.2 to 4 wt. %, preferably 0.33 to 3 wt. %, and particularly preferably 0.5 to 2 wt. %, each converted to the undissociated acid and based on the weight of the treatment agent.

Even if the dicarboxylic acids are present in salt form, the above quantity information relates to the respective dicarboxylic acid in undissociated form so as not to distort the quantity information by different molecular weights of the salts. An initial weight of 15 wt. % disodium succinate hexahydrate, for example, would result in an equivalent concentration of succinic acid of 6.55 wt. %.

Amino Acids

The reduced damaging effect on the hair by the treatment agents according to the invention can essentially be attributed to the at least one aforementioned dicarboxylic acid in cooperation with at least one amino acid.

The treatment agents according to the invention thus contain at least one amino acid as a further mandatory component. Preferred amino acids are selected from arginine, lysine, histidine, asparagine, glutamine, cysteine, methionine, tryptophan, serine, alanine, aspartic acid, glutamic acid, glycine, isoleucine, leucine, phenylalanine, proline, threonine, tyrosine and valine, and mixtures of these amino acids. Further preferred oxidative treatment agents according to the invention contain at least one amino acid in a total amount of 0.05 to 3 wt. %, preferably 0.1 to 2 wt. %, and particularly preferably 0.2 to 1.2 wt. %, each converted to the undissociated acid and based on the weight of the oxidative treatment agent. Further preferred oxidative treatment agents according to the invention contain at least one amino acid, selected from arginine, lysine, histidine, asparagine, glutamine, cysteine, methionine, tryptophan, serine, alanine, aspartic acid, glutamic acid, glycine, isoleucine, leucine, phenylalanine, proline, threonine, tyrosine and valine, and mixtures of these amino acids, in a total amount of 0.05 to 3 wt. %, preferably 0.1 to 2 wt. %, and particularly preferably 0.2 to 1.2 wt. %, each converted to the undissociated acid and based on the weight of the oxidative treatment agent.

Amino Acid of Formula (VI)

A particularly drastically reduced damaging effect on the hair by the treatment agents according to the invention can essentially be attributed to the aforementioned dicarboxylic acids in cooperation with at least one selected amino acid of formula (VI).

Preferred oxidative treatment agents according to the invention thus comprise, as a mandatory component, at least one amino acid of formula (VI)

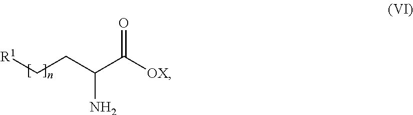

where
X denotes a hydrogen atom or a monovalent or divalent cation;
n denotes zero, 1, 2 or 3;
R¹ denotes a group selected from an amino group, a guanidine group, a (1H-imidazole-4-yl) group, a carboxylic acid amid group —CONH₂, a 1H-indole-3-yl group, a thiol group —SH, and a methylsulfanyl group —SCH₃, or at least one salt of these amino acids.

Preferred amino acids of formula (VI) are selected from arginine, lysine, histidine, asparagine, glutamine, cysteine, methionine, tryptophan, and mixtures thereof. Particularly preferred oxidative treatment agents contain mixtures of arginine and lysine, or at least one salt of these amino acids.

Preferred oxidative treatment agents according to the invention contain the at least one amino acid of formula (VI), or one or more salts thereof, in a total amount of 0.05 to 3 wt. %, preferably 0.1 to 2 wt. %, and particularly preferably 0.2 to 1.2 wt. %, each converted to the undissociated acid and based on the weight of the treatment agent. Further particularly preferred treatment agents according to the invention contain mixtures of arginine and lysine, or at least one salt of these amino acids, in a total amount of 0.05 to 3 wt. %, preferably 0.1 to 2 wt. %, and particularly preferably 0.2 to 1.2 wt. %, each converted to the undissociated acid and based on the weight of the treatment agent.

Hydrogen Peroxide

The formation of the dyes in oxidative coloring agents, or the depletion of melanin, the pigment that gives hair its color, for blonding requires the influence of a peroxide compound as an oxidizing agent. Usually, hydrogen peroxide is used for this purpose. Hydrogen peroxide can only be used in the form of an aqueous solution.

Preferred oxidative treatment agents according to the invention are characterized by comprising 0.5 to 13 wt. %, more preferably 1 to 7 wt. %, particularly preferably 2 to 6 wt. %, and especially particularly preferably 3 to 4.5 wt. % hydrogen peroxide (calculated as 100% H₂O₂), in each case based on the total weight of the oxidative treatment agent according to the invention.

Blonding agents, or coloring agents that have a particularly strong lightening effect, can additionally comprise strongly oxidizing peroxide compounds, such as potassium, sodium and/or ammonium persulfate and/or sodium percarbonate.

Water

The oxidative treatment agents according to the invention comprise water, in particular preferably in an amount of 20 to 95 wt. %, preferably 30 to 90 wt. %, particularly preferably 40 to 85 wt. %, and exceptionally preferably 50 to 80 wt. %, in each case based on the total weight of the oxidative treatment agent according to the invention.

pH Value

Oxidative treatment agents according to the invention have a pH value in the range of 2.5 to 6.5, preferably in the range of 3 to 5.5, and particularly preferably in the range of 3.5 to 5.0, each measured at 20° C.,

Chelating Agent

Surprisingly, the effect according to the invention can be enhanced when the oxidative treatment agent comprises at least one chelating agent. According to the invention, a chelating agent shall be understood to mean a compound that forms chelates with calcium ions or magnesium ions in an aqueous solution. According to the invention, chelates shall be understood to mean cyclic compounds in which metal ions, furthermore groups including lone electron pairs or having electron gaps, and furthermore hydrogen, are involved in the ring formation.

Preferred chelating agents according to the invention have a molecular weight of 100 g/mol to 600 g/mol. Further preferred chelating agents according to the invention are selected from pyridine-2,6-dicarboxylic acid (dipicolinic acid), 1-hydroxyethane-1,1-diphosphonic acid (HEDP, etidronic acid) and the sodium, potassium and ammonium salts thereof, disodium pyrophosphate, beta-alanine diacetic acid, ethylenediaminetetraacetic acid (EDTA) and the sodium, potassium and ammonium salts thereof, aminotrimethylene phosphonic acid, diethylenetriamine pentamethylene phosphonic acid, disodium azacycloheptane diphosphonate, disodium hydroxyethyl-amino-bis-methylphosphonate, disodium hydroxyethyliminodiacetate, hydroxyethyl ethylenediaminetriacetic acid (HEDTA) and the sodium or potassium salts thereof, pentapotassium triphosphate, pentasodium triphosphate, pentasodium aminotrimethylene phosphonate, pentasodium ethylenediamine tetramethylene phosphonate, pentasodium diethylenetriaminepentaacetate (pentasodium pentetate), diethylenetriaminepentaacetic acid (pentetic acid), phosphonobutane tricarboxylic acid, potassium ethylenediamine tetramethylene phosphonate (potassium EDTMP), sodium ethylenediamine tetramethylene phosphonate (sodium EDTMP), sodium diethylenetriamine pentamethylene phosphonate (sodium DTPMP), sodium hexametaphosphate, tetrapotassium pyrophosphate, tetrasodium pyrophosphate, tetrasodium dicarboxymethyl aspartate, tetrasodium iminodisuccinate, trisodium dicarboxymethyl alaninate, trisodium ethylenediamine disuccinate (trisodium EDDS) and trisodium nitrilotriacetate, and mixtures of these chelating agents.

Particularly preferred chelating agents according to the invention are selected from pyridine-2,6-dicarboxylic acid (dipicolinic acid), 1-hydroxyethane-1,1-diphosphonic acid (HEDP, etidronic acid) and the sodium, potassium and ammonium salts thereof, disodium pyrophosphate, ethylenediaminetetraacetic acid (EDTA) and the potassium and ammonium salts thereof, pentasodium diethylenetriaminepentaacetate (pentasodium pentetate), tetrasodium pyrophosphate, trisodium ethylenediamine disuccinate (trisodium EDDS) and mixtures of these chelating agents.

Particularly preferred oxidative treatment agents according to the invention comprise at least one chelating agent in a total amount of 0.01 to 2.5 wt. %, preferably in a total amount of 0.1 to 1.5 wt. %, and particularly preferably in a total amount of 0.2 to 0.6 wt. %, in each case based on the weight of the oxidative treatment agent.

Surprisingly, it was furthermore found that the reduced damaging effect on the hair by the oxidative treatment agents according to the invention and the preferred oxidative treatment agents according to the invention can be further supported if at least one compound of the general formula (III) is present.

Preferred oxidative treatment agents according to the invention thus comprise
(a) at least one compound of the general formula (III),

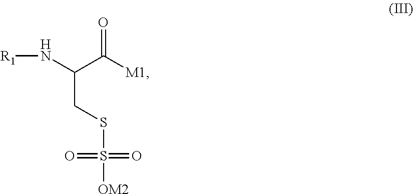

wherein
R1 denotes a hydrogen atom or a structural element of formula (IV)

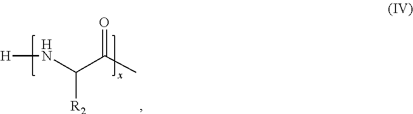

wherein
a denotes an integer from 1 to 100,
the group R2 in each of the structural elements of formula (IV) can each be selected independently of the preceding structural element of formula (IV),
R2 denotes a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazole-4-ylmethyl group, a 1H-indole-3-ylmethyl group or a (sulfosulfanyl)methyl group,
M1 denotes the group —OM2 or a structural element of formula (V),

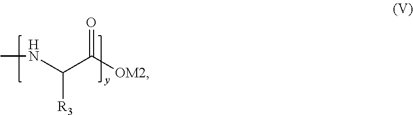

wherein
y denotes an integer from 1 to 100,
the group R3 in each of the structural elements of formula (V) can each be selected independently of the preceding structural element of formula (V),
R3 denotes a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazole-4-ylmethyl group, a 1H-indole-3-ylmethyl group or a (sulfosulfanyl)methyl group,
M2 denotes a hydrogen atom, an equivalent of a monovalent or polyvalent cation, or an ammonium ion (NH₄)⁺.

The essential ingredient (a) of formula (III) is the Bunte salt of an amino acid, an oligopeptide or a peptide, which represents a compound of formula (III),

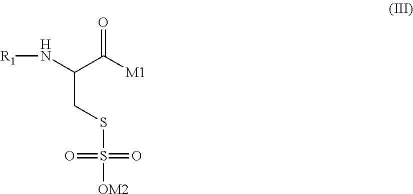

wherein
R1 denotes a hydrogen atom or a structural element of formula (IV)

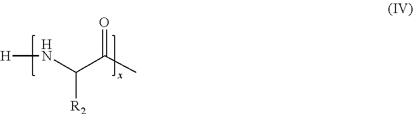

wherein
a denotes an integer from 1 to 100,
the group R2 in each of the structural elements of formula (IV) can each be selected independently of the preceding structural element of formula (IV),
R2 denotes a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazole-4-ylmethyl group, a 1H-indole-3-ylmethyl group or a (sulfosulfanyl)methyl group,
M1 denotes the group —OM2 or a structural element of formula (V),

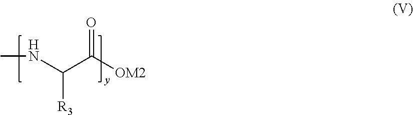

wherein
y denotes an integer from 1 to 100,
the group R3 in each of the structural elements of formula (V) can each be selected independently of the preceding structural element of formula (V),
R3 denotes a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazole-4-ylmethyl group, a 1H-indole-3-ylmethyl group or a (sulfosulfanyl)methyl group,
M2 denotes a hydrogen atom, an equivalent of a monovalent or polyvalent cation, or an ammonium ion (NH₄)⁺.

The group R1 can either denote a hydrogen atom or a structural element of formula (IV)

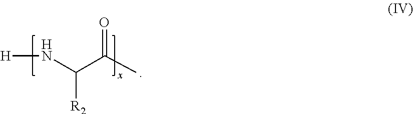

The structural element of formula (IV) is furthermore characterized by the repetition index x, wherein x denotes an integer from 1 to 100. The repetition index x indicates the number of structural elements of formula (IV) present in the compound of formula (III).

The letter x preferably denotes an integer from 1 to 50, more preferably x denotes an integer from 1 to 20, and especially particularly preferably x denotes an integer from 1 to 10.

If x denotes the number 10, for example, the compound of formula (III) comprises 10 structural elements of formula (IV).

What is essential here is that the group R2 in each of the structural elements of formula (IV) can each be selected independently of the preceding structural element of formula (IV). If the compounds of formula (III) comprise 10 structural units of formula (IV), for example, these 10 structural units may be identical, or else they may be different from each other.

The group R2 denotes a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazole-4-ylmethyl group, a 1H-indole-3-ylmethyl group or a (sulfosulfanyl)methyl group.

The structural element of formula (IV) is thus an amino acid that is peptidically linked via the amino function thereof and/or the acid function thereof within the compound of formula (III). If the amino acid is cysteine, this may also be present in the form of a Bunte salt.

If the group R2 denotes a hydrogen atom, the structural element of formula (IV) is based on the amino acid glycine.

If the group R2 denotes a methyl group, the structural element of formula (IV) is based on the amino acid alanine.

If the group R2 denotes an isopropyl group (which is to say a (H₃C)₂CH— group), the structural element of formula (IV) is based on the amino acid valine.

If the group R2 denotes a 2-methylpropyl group (which is to say a (H₃C)₂CH—CH₂— group), the structural element of formula (IV) is based on the amino acid leucine.

If the group R2 denotes a 1-methylpropyl group (which is to say a (H3C)2CH—CH2- group), the structural element of formula (IV) is based on the amino acid isoleucine.

If the group R2 denotes a benzyl group (which is to say a C₆H₅—CH₂— group), the structural element of formula (IV) is based on the amino acid phenylalanine.

If the group R2 denotes a 4-hydroxybenzyl group (which is to say a 4-OH—C₆H₅—CH₂— group), the structural element of formula (IV) is based on the amino acid tyrosine.

If the group R2 denotes a hydroxymethyl group (which is to say a (H3C)2CH— group), the structural element of formula (IV) is based on the amino acid serine.

If the group R2 denotes a 1-hydroxyethyl group (which is to say a (H3C—CH(OH)— group), the structural element of formula (IV) is based on the amino acid threonine.

If the group R2 denotes a 4-aminobutyl group (which is to say a H2N—CH2-CH2-CH2-CH2- group), the structural element of formula (IV) is based on the amino acid lysine.

If the group R2 denotes a 3-carbamimidamidopropyl group (which is to say a H₂N—C(NH)—NH—CH₂—CH₂—CH₂— group), the structural element of formula (IV) is based on the amino acid arginine.

If the group R2 denotes a 2-carboxyethyl group (which is to say a HOOC—CH2-CH2- group), the structural element of formula (IV) is based on the amino acid glutamic acid.

If the group R2 denotes a carboxymethyl group (which is to say a HOOC—CH2- group), the structural element of formula (IV) is based on the amino acid aspartic acid.

If the group R2 denotes a 2-carbamoylethyl group (which is to say a H2N—C(O)—CH2-CH2- group), the structural element of formula (IV) is based on the amino acid glutamine.

If the group R2 denotes a 2-carbamoylethyl group (which is to say a H2N—C(O)—CH2- group), the structural element of formula (IV) is based on the amino acid asparagine.

If the group R2 denotes a sulfanylmethyl group (which is to say a HS—CH2- group), the structural element of formula (IV) is based on the amino acid cysteine.

If the group R2 denotes a 2-(methylsulfanyl)ethyl group (which is to say a H3C—S—CH2-CH2- group), the structural element of formula (IV) is based on the amino acid methionine.

If the group R2 denotes a 1H-imidazole-4-ylmethyl group, the structural element of formula (IV) is based on the amino acid histidine.

If the group R2 denotes a 1H-indole-3-ylmethyl group, the structural element of formula (IV) is based on the amino acid tryptophan.

Finally, the group R2 may also denote a (sulfosulfanyl)methyl group, which involves a Bunte salt structure of formula HO—S(O₂)—S—CH₂—.

Depending on the pH value of the treatment agent, the Bunte salt structure of formula HO—S(O₂)—S—CH₂— may also be present in the deprotonated form thereof.

Within the compound of formula (III), M1 denotes the group —OM2 or a structural element of formula (V)

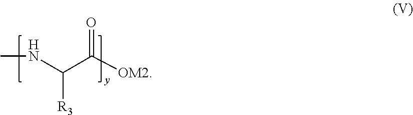

The structural element of formula (V) is characterized by the repetition index y, wherein y denotes an integer from 1 to 100. The repetition index y indicates the number of structural elements of formula (V) present in the compound of formula (III).

The letter y preferably denotes an integer from 1 to 50, more preferably y denotes an integer from 1 to 20, and especially particularly preferably y denotes an integer from 1 to 10.

If y denotes the number 10, for example, the compound of formula (III) comprises 10 structural elements of formula (V).

What is essential here is that the group R3 in each of the structural elements of formula (V) can each be selected independently of the preceding structural element of formula (V). If the compounds of formula (III) comprise 10 structural units of formula (V), for example, these 10 structural units may be identical, or else they may be different from each other.

The group R3 denotes a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazole-4-ylmethyl group, a 1H-indole-3-ylmethyl group or a (sulfosulfanyl)methyl group.

The structural element of formula (V) is thus also an amino acid that is peptidically linked via the amino function thereof and/or the acid function thereof within the compound of formula (III). If the amino acid is cysteine, this may also be present in the form of a Bunte salt.

If the group R3 denotes a hydrogen atom, the structural element of formula (V) is based on the amino acid glycine.

If the group R3 denotes a methyl group, the structural element of formula (V) is based on the amino acid alanine.

If the group R3 denotes an isopropyl group (which is to say a (H₃C)₂CH— group), the structural element of formula (V) is based on the amino acid valine.

If the group R3 denotes a 2-methylpropyl group (which is to say a (H₃C)₂CH—CH₂— group), the structural element of formula (V) is based on the amino acid leucine.

If the group R3 denotes a 1-methylpropyl group (which is to say a (H3C)2CH—CH2- group), the structural element of formula (V) is based on the amino acid isoleucine.

If the group R3 denotes a benzyl group (which is to say a C₆H₅—CH₂— group), the structural element of formula (V) is based on the amino acid phenylalanine.

If the group R3 denotes a 4-hydroxybenzyl group (which is to say a 4OH—C₆H₅—CH₂— group), the structural element of formula (V) is based on the amino acid tyrosine.

If the group R3 denotes a hydroxymethyl group (which is to say a (H3C)2CH— group), the structural element of formula (V) is based on the amino acid serine.

If the group R3 denotes a 1-hydroxyethyl group (which is to say a (H3C—CH(OH)— group), the structural element of formula (V) is based on the amino acid threonine.

If the group R3 denotes a 4-aminobutyl group (which is to say a H2N—CH2-CH2-CH2-CH2- group), the structural element of formula (V) is based on the amino acid lysine.

If the group R3 denotes a 3-carbamimidamidopropyl group (which is to say a H₂N—C(NH)—NH—CH₂—CH₂—CH₂— group), the structural element of formula (V) is based on the amino acid arginine.

If the group R3 denotes a 2-carboxyethyl group (which is to say a HOOC—CH2-CH2- group), the structural element of formula (V) is based on the amino acid glutamic acid.

If the group R3 denotes a carboxymethyl group (which is to say a HOOC—CH2- group), the structural element of formula (V) is based on the amino acid aspartic acid.

If the group R3 denotes a 2-carbamoylethyl group (which is to say a H2N—C(O)—CH2-CH2- group), the structural element of formula (V) is based on the amino acid glutamine.

If the group R3 denotes a 2-carbamoylethyl group (which is to say a H2N—C(O)—CH2- group), the structural element of formula (V) is based on the amino acid asparagine.

If the group R3 denotes a sulfanylmethyl group (which is to say a HS—CH2- group), the structural element of formula (V) is based on the amino acid cysteine.

If the group R3 denotes a 2-(methylsulfanyl)ethyl group (which is to say a H3C—S—CH2-CH2- group), the structural element of formula (V) is based on the amino acid methionine.

If the group R3 denotes a 1H-imidazole-4-ylmethyl group, the structural element of formula (V) is based on the amino acid histidine.

If the group R3 denotes a 1H-indole-3-ylmethyl group, the structural element of formula (V) is based on the amino acid tryptophan.

Finally, the group R3 may also denote a (sulfosulfanyl)methyl group, which involves a Bunte salt structure of formula HO—S(O₂)—S—CH₂—.

Depending on the pH value of the treatment agent, the Bunte salt structure of formula HO—S(O₂)—S—CH₂— may also be present here in the deprotonated form thereof.

The group M2 denotes a hydrogen atom, an equivalent of a monovalent or polyvalent cation, or an ammonium ion (NH₄)⁺.

In particular, the cations of sodium and potassium (Na⁺ and K⁺) or else of magnesium or calcium (½ Mg²⁺ or ½ Ca²⁺) may be mentioned as preferred equivalents of a monovalent or polyvalent cation.

If M2 denotes a hydrogen atom, the —OM2 group involves the OH group. If M2 denotes a sodium cation, the —OM2 group involves the —ONa group. If M2 denotes a potassium cation, the —OM2 group involves the —OK group. If M2 denotes an ammonium ion, the —OM2 group involves the —O(NH₄) group.

The —OM2 group always adjoins a carbonyl group. In sum, if M2 denotes H, K, Na or ammonium, in the compound of formula (III) is thus either present in the form of an acid in the protonated form thereof, or the sodium, potassium or ammonium salt of this acid.

The compounds of formula (III) according to the invention are either the Bunte salt of the amino acid cysteine, the Bunte salts of oligopeptides, or else the Bunte salts of peptides.

When R1 denotes a hydrogen atom and the group M1 denotes an —OM2 group, the compound of formula (III) is the Bunte salt of the amino acid cysteine. In this case, the compound of formula (III) is the compound of formula (IIIa),

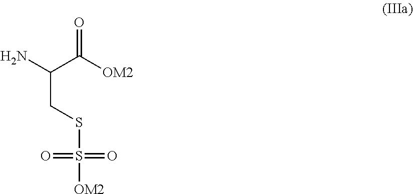

wherein M2 is again defined as described above.

If the compound of formula (IIIa) is present in the form of the free acid thereof, then this involves 2-amino-3-(sulfosulfanyl)propane acid. This substance is commercially available.

It was found that the use of the compound of formula (IIIa) in treatment agents results in a particularly effective reduction in damage to the hair already at particularly low amounts, and that this reduction is still present even after washing the hair several times. The use of compounds of formula (IIIa) in treatment agents is therefore especially particularly preferred.

In an especially particularly preferred embodiment, an oxidative treatment agent according to the invention is characterized by comprising at least one compound of formula (III), wherein
R1 denotes a hydrogen atom, and
M1 denotes an —OM2 group.

When a compound of formula (IIIa) is used, this preferably involves the use of this specific compound. However, if the Bunte salts of oligopeptides are used as compounds of formula (III), the treatment agent according to the invention can also comprise several compounds of formula (III) as a mixture of different oligopeptides. These oligopeptides are defined by the average molecular weight thereof. The average molecular weight M_{w} of the at least one oligopeptide of formula (III) can be determined, for example, by way of gel permeation chromatography (GPC) using polystyrene as the internal standard in accordance with DIN 55672-3, Version 8/2007.

Depending on the number of structural elements of formula (III) and/or (IV) present in the compound of formula (III), and depending on the type of these amino acids, the molecular weight of the compound of formula (III) used according to the invention may vary. It is particularly preferred according to the invention if the compound of formula (III) is an oligopeptide that has a molecular weight M_{w} of 200 to 2,000 Da, especially of 250 to 1,500 Da, preferably of 300 to 1.200 Da, and in particular of 400 to 800 Da.

The term oligopeptide within the scope of the present invention shall be understood to mean condensation products of amino acids that have the above-described molecular weights.

In an especially particularly preferred embodiment, an oxidative treatment agent according to the invention is characterized by comprising at least one compound of formula (III) that has a molecular weight M_{w} of 200 to 2,000 Da (Dalton), especially of 250 to 1,500 Da, preferably of 300 to 1,200 Da, and in particular of 400 to 800 Da.

If a mixture of oligomers is used in the treatment agent according to the invention, these mixtures may also be defined by way of the average molecular weights thereof.

In this case, a preferred treatment agent according to the invention is characterized by comprising at least one mixture of compounds of formula (III) that has an average molecular weight M_{w} of 200 to 2,000 Da, especially of 250 to 1,500 Da, preferably of 300 to 1,200 Da, and in particular of 400 to 800 Da.

Furthermore, it was found that the protective or repair effect of the compounds of formula (III) is also dependent on the repetition indices x and y. As described above, it is especially particularly preferred when x denotes an integer from 1 to 10, and y denotes an integer from 1 to 10.

In a further especially particularly preferred embodiment, an oxidative treatment agent according to the invention is characterized by comprising at least one compound of formula (III), wherein
R1 denotes a structural element of formula (IV), and
M1 denotes a structural element of formula (V), and
x denotes an integer from 1 to 10; and
y denotes an integer from 1 to 10.

In addition to the molecular weight of the compound of formula (III), the content of the Bunte salts units present in the compound of formula (III) also decisively influences the effectiveness of the protective action or “repair action” of the compounds.

Compounds having at least one Bunte salt unit, such as is present in the compound of formula (IIIa), for example, are very effective, in particular when they are used as a monomeric compound. Oligopeptides having at least one Bunte salt unit are particularly effective when they have a low molecular weight of up to 1200 dalton, and in particular 800 dalton.

When oligopeptides are used, however, it is especially particularly advantageous when the compound of formula (III) includes at least two, and preferably at least three, Bunte salt units.

In a further especially particularly preferred embodiment, a treatment agent according to the invention is characterized by comprising at least one compound of formula (III), wherein
R1 denotes a structural element of formula (IV), and
the group R2 denotes a (sulfosulfanyl)methyl group (which is to say a HO—S(O₂)—S—CH₂— group) in at least one structural element of formula (IV).

In a further especially particularly preferred embodiment, a treatment agent according to the invention is characterized by comprising at least one compound of formula (III), wherein
R1 denotes a structural element of formula (IV), and
x denotes an integer of at least 3; and
the group R2 denotes a 2-carboxyethyl group (which is to say a HOOC—CH2-CH2- group) in at least 3 structural elements of formula (IV).

In a further especially particularly preferred embodiment, a treatment agent according to the invention is characterized by comprising at least one compound of formula (III), wherein
M1 denotes a structural element of formula (V), and
y denotes an integer of at least 3, and
the group R3 denotes a (Glu) group in at least 3 structural elements of formula (IV).

The at least one compound of formula (III) is present in a total amount of 0.001 to 10 wt. %, based on the total weight of the preferred treatment agent according to the invention. Surprisingly, however, it was found that the compound(s) of formula (III) can bring about an excellent reduction in damage to the hair already in low concentrations. This is in particular advantageous when the at least one compound of formula (III) is to be added to the treatment agent according to the invention as an additive (for example in the form of a nourishing lotion or repair lotion) prior to being applied to the hair. For this reason, it is particularly advantageous if the preferred oxidative treatment agent according to the invention comprises one or more compounds of the above-described formula (III) in a total amount of 0.001 to 2.5 wt. %, more preferably of 0.01 to 1.0 wt. %, and particularly preferably of 0.02 to 0.1 wt. %, in each case based on the weight of the oxidative treatment agent according to the invention.

In a further especially particularly preferred embodiment, an oxidative treatment agent according to the invention is characterized by comprising one or more compounds of the above-described formula (III) in a total amount of 0.001 to 2.5 wt. %, more preferably of 0.01 to 1.0 wt. %, and particularly preferably of 0.02 to 0.1 wt. %, in each case based on the weight of the treatment agent according to the invention.

Fatty Acid Amides

Surprisingly, the effect according to the invention can be enhanced when the oxidative treatment agent comprises at least one fatty acid amide of formula (AMID-I), as described hereafter. Preferred oxidative treatment agents according to the invention comprise at least one fatty acid amide of formula (AMID-I),

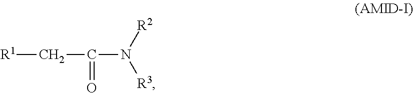

where
R¹ is selected from
linear or branched C4 to C22 alkyl groups, which are saturated or unsaturated, wherein the alkyl and alkenyl groups are unsubstituted or may be substituted with oxygen atoms, nitrogen atoms, hydroxyl groups, ether groups, oxyalkylene groups, polyoxyalkylene groups, carboxylic acid groups, amine groups, amide groups, halogen-containing groups, ester groups, siloxane groups, or polysiloxane groups,
and from alkoxylated alkyl groups of formulas

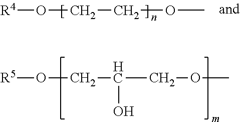

where:
R⁴ and R⁵, which can be the same or different, are selected from linear or branched C4 to C22 alkyl groups, which are saturated or unsaturated,
n is an integer in the range from 1 to 10, and preferably in the range from 1 to 4; and
m is an integer in the range from 1 to 6; and
R² and R³, which may be the same or different, are each independently of one another selected from H; linear or branched C1 to C22 alkyl groups, which are saturated or unsaturated, wherein the alkyl and alkenyl groups are unsubstituted or substituted with oxygen atoms, nitrogen atoms, hydroxyl groups, ether groups, oxyalkylene groups, and in particular oxyethylene groups, polyoxyalkylene groups, and in particular polyoxyethylene groups, carboxylic acid groups, amine groups, alkylamine groups, and in particular C1 to C3 alkylamine groups, dialkylamine groups, and in particular di-(C1-C3)-alkylamine groups, substituted and non-substituted 4,5-dihydroimidazole groups, amide groups, halogen-containing groups, ester groups, siloxane groups, or polysiloxane groups.

Preferably, at least one fatty acid amide of formula (AMID-I), as represented above, is present in a total amount of 0.1 to 10 wt. %, particularly preferably 0.2 to 5 wt. %, and exceptionally preferably 0.4 to 2 wt. %, in each case based on the weight of the oxidative treatment agent.

Particularly preferred fatty acid amides of formula (AMID-I) are those in which R¹ is selected from alkoxylated alkyl groups of formula

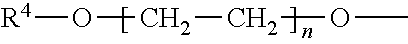

where n is an integer in the range from 1 to 10, and preferably in the range from 1 to 4, R⁴ is selected from a saturated linear C4 to C22 alkyl group, R² is hydrogen, and R³ selected from a saturated linear C1 to C22 alkyl group that is substituted with a hydroxyl group. Further particularly preferred fatty acid amides of formula (AMID-I) are those in which R¹ is selected from alkoxylated alkyl groups of formula

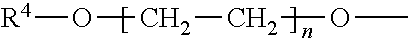

where n is 1, R⁴ is selected from a saturated linear C11 to C16 alkyl group, R² is hydrogen, and R³ is selected from a beta-hydroxyethyl group. Such a particularly preferred fatty acid amide according to the invention is trideceth-2 carboxamide MEA, for example.

Further particularly preferred fatty acid amides of formula (AMID-I) are those in which R¹ is selected from a saturated linear C11 to C21 alkyl group, R² is hydrogen, and R³ is selected from a saturated linear C1 to C22 alkyl group that is substituted with an amine group, a C1 to C3 alkyl amine group, or a di-(C1-C3)-alkylamine group. Further particularly preferred fatty acid amides of formula (AMID-I) are those in which R¹ is selected from a saturated linear C11 to C21 alkyl group, R² is hydrogen, and R³ is selected from a gamma-dimethylamine-n-propyl group. Such particularly preferred fatty acid amides according to the invention are stearamidopropyl dimethylamine and behenamidopropyl dimethylamine, for example.

Further particularly preferred fatty acid amides of formula (AMID-I) are those in which R¹ is selected from a saturated linear C11 to C21 alkyl group, R² is hydrogen, and R³ is selected from a saturated linear C1 to C3 alkyl group that is substituted in the omega position with a 4,5-dihydroimidazolium group, wherein the latter is substituted at the 2-position with a C8 to C22 alkyl group and at the 1-position with a methyl group. Further particularly preferred fatty acid amides of formula (AMID-I) are those in which R¹ is selected from a saturated linear C11 to C21 alkyl group, R² is hydrogen, and R³ is selected from an ethyl group that is substituted in the omega position with a 4,5-dihydroimidazolium group, wherein the latter is substituted at the 2-position with a C8 to C22 alkyl group and at the 1-position with a methyl group. Such particularly preferred fatty acid amides according to the invention are, for example, 1-(docosanamidoethyl)-2-heneicosyl-1-methyl-4,5-dihydroimidazolium methyl sulfate, which bears the INCI name Quaternium-91, and 1-(cocoylamidoethyl)-2-cocoyl-1-methyl-4, 5-dihydroimidazolium methyl sulfate, which bears the INCI name Quaternium-87 and the CAS number 92201-88-2. Particularly preferred oxidative treatment agents according to the invention comprise, in each case based on the weight thereof, a total amount of 0.1 to 10 wt. %, particularly preferably 0.2 to 5 wt. %, and exceptionally preferred 0.4 to 2 wt. % of at least one of the fatty acid amides of formula (AMID-I), including at least one compound that is selected from trideceth-2 carboxamide MEA, stearamidopropyl dimethylamine, behenamidopropyl dimethylamine, Quaternium-87, and Quaternium-91.

The oxidative treatment agents according to the invention can be applied directly and without any further composition to the keratin fibers to be treated, and in particular to the human hair. This type of application is in particular suitable for achieving minor lightening or blonding of the keratin fibers and/or for preserving the lightening effect of a prior blonding process for a longer period. However, so as to achieve a stronger lightening or blonding action, the oxidative treatment agent according to the invention must be set to a pH value in the range of 6 or 6.5 to 12 (measured at 20° C.) by adding an alkalizing agent. Since alkaline hydrogen peroxide solutions are not storage stable, the alkalizing agent is added just prior to the application to the keratin fibers. These alkalizing agents, hereafter referred to as composition (B), may be solid, and in particular powdery, but also liquid (0 to 500 mPas at 20° C.), of medium viscosity (>400 to 4000 mPas at 20° C.), or creamy or pasty (>4000 to 4,000,000 mPas at 20° C.). If composition (B) is powdery, it is preferred according to the invention for the same to comprise at least one persulfate salt and/or at least one percarbonate salt.

The alkalizing agent-containing composition (B) can either be anhydrous or hydrous. If the mixture made of the oxidative treatment agent according to the invention and the alkalizing agent-containing composition (B) is intended to be used for lightening or blonding keratin fibers, and in particular human hair, composition (B) usually does not include any oxidation dye precursors. However, it may include certain direct dyes, which compensate for an undesirable red or orange undertone of the decomposition products of melanin. This technique is disclosed in WO 2002/074270 A1 or in EP 2306963 A1, for example.

Alkalizing Agent for Composition (B)

The compositions (B) suitable for being combined with the oxidative treatment agent according to the invention comprise at least one alkalizing agent, selected from ammonium hydroxide, monoethanolamine and sodium silicates, and mixtures thereof.

So as to achieve the desired permanent coloration or lightening of the keratin fibers, composition (B), which is mixed with the oxidative treatment agent according to the invention to yield the ready-to-use coloring or lightening agent, must have a pH value in the range of 6.5 to 11.0, preferably 8 to 10.5, and particularly preferably 8.5 to 10, each measured at 20° C. Furthermore, it is important for the coloring or lightening result for the ready-to-use coloring or lightening agent, which is yielded by mixing the oxidative treatment agent according to the invention with composition (B), to have a pH value in the range of 6.5 to 11.0, preferably 8 to 10.5, and particularly preferably 8.5 to 9.5, each measured at 20° C. At these pH values, the outer keratin fiber layer opens optimally to absorb the oxidation dye precursors and/or the oxidative action of the hydrogen peroxide, and optionally of further peroxide compounds, develops optimally.

Ammonia is preferably used in the form of the aqueous solution thereof. Corresponding aqueous ammonia solutions may be 10 to 35 percent solutions (calculated in wt. %, 100 g aqueous ammonia solution accordingly comprises 10 to 35 g ammonia). Ammonia is preferably used in the form of a 20 to 30 wt. % solution, and particularly preferably in the form of a 25 wt. % solution.

In a particularly preferred embodiment, composition (B) used according to the invention is characterized by comprising ammonium hydroxide in an amount of 0.20 to 18.0 wt. %, preferably of 1.0 to 15 wt. %, more preferably of 2.0 to 12.0 wt. %, and particularly preferably of 3.0 to 9 wt. %, based on the weight of composition (B) used according to the invention.

In addition to, or instead of, ammonium hydroxide, preferred compositions (B) used according to the invention comprise monoethanolamine.

So as to achieve maximum odor masking and optimize the fastness properties, monoethanolamine is present in a total amount of 0.2 to 9.0 wt. %, preferably of 1.0 to 7 wt. %, more preferably of 2.0 to 6.0 wt. %, and particularly preferably of 3.0 to 5.5 wt. %, based on the weight of composition (B) used according to the invention.

Sodium silicates within the meaning of the present invention are chemical compounds that are composed of sodium oxide and silicon dioxide and can be present in various molar ratios (monosilicate, metasilicate and polysilicate). One example of a sodium silicate is the sodium salt of orthosilicic acid having the empirical formula Na₄SiO₄, which is also referred to as sodium orthosilicate.

Further examples of suitable sodium silicates are the disodium metasilicate or sodium metasilicate having the empirical formula Na₂SiO₃, the disodium disilicate having the empirical formula Na₂Si₂O₅, or the disodium trisilicate having the empirical formula Na₂Si₃O₇.

Silicates in amorphous form can be produced by fusing silicon dioxide and alkali oxide in molar ratios between 1:1 and 4:1. The solids thus obtained are dissolved at approximately 150° C. and a vapor pressure of 5 bar so as to obtain a solution of the sodium silicates in water; these corresponding solutions are alkali sodium silicates.

Alkali sodium silicates is the term used for vitreous (amorphous) sodium silicates or the aqueous solutions thereof, solidified from a melt. These are also referred to as sodium water glasses. The definition of the sodium silicates within the present invention also covers sodium water glasses.

The molar composition of water glasses is usually 2 to 4 moles SiO₂ for 1 mole alkali oxide (Na₂O).

One example of a preferred sodium silicate is sodium water glass that is present in the form of the aqueous solution thereof, has a Na₂O content of 7.5 to 8.8 wt. %, and an SiO₂ content of 25.0 to 28.5 wt. %, and has the CAS number 1344-09-5 (Chemical Abstracts number).

Further compositions (B) used preferably according to the invention include at least one sodium silicate in a total amount of 0.1 to 9 wt. %, preferably 0.2 to 8 wt. %, and particularly preferably 1 to 7.5 wt. %, in each case based on the weight of composition (B) used according to the invention.

Furthermore, other alkalizing agents, such as potassium hydroxide (KOH) and sodium hydroxide (NaOH), may be present, typically in a total amount of 0.05 to 1.5 wt. %, and preferably 0.1 to 0.6 wt. %, in each case based on the weight of composition (B) used according to the invention.

If the mixture made of the oxidative treatment agent according to the invention and the alkalizing agent-containing composition (B) is intended to be used for oxidatively coloring keratin fibers, and in particular human hair, composition (B) comprises at least one oxidation dye precursor for forming the dyes.

Oxidation dye precursors cover oxidation dye precursors of the developer type and of the coupler type. Particularly suitable oxidation dye precursors of the developer type are selected from at least one compound from the group consisting of p-phenylenediamine, p-toluylenediamine, 2-(2-hydroxyethyl)-p-phenylenediamine, 2-(1,2-dihydroxyethyl)-p-phenylenediamine, N,N-bis-(2-hydroxyethyl)-p-phenylenediamine, 2-methoxymethyl-p-phenylenediamine, N-(4-amino-3-methylphenyl)-N-[3-(1H-imidazole-1-yl)propyl]amine, N,N′-bis-(2-hydroxyethyl)-N,N′-bis-(4-aminophenyl)-1,3-diamino-propane-2-ol, bis(2-hydroxy-5-aminophenyl)methane, 1,3-bis-(2,5-diaminophenoxy)-propane-2-ol, N,N′-bis-(4-aminophenyl)-1,4-diazacycloheptane, 1,10-bis-(2,5-diaminophenyl)-1,4,7,10-tetraoxadecane, p-aminophenol, 4-amino-3-methylphenol, 4-amino-2-aminomethylphenol, 4-amino-2-(1,2-dihydroxyethyl)phenol, 4-amino-2-(diethyl aminomethyl)phenol, 4,5-diamino-1-(2-hydroxyethyl)pyrazole, 2,4,5,6-tetraaminopyrimidine, 4-hydroxy-2,5,6-triaminopyrimidine, 2-hydroxy-4,5,6-triaminopyrimidine, 2,3-diamino-6,7-dihydro-1H,5H-pyrazolo [1,2-a]pyrazole-1-on, and the physiologically compatible salts thereof.

Particularly suitable oxidation dye precursors of the coupler type are selected from the group consisting of 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)-amino-2-methylphenol, 2,4-dichloro-3-aminophenol, 2-aminophenol, 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4, 5-dimethylphenyl}amino)ethanol, 2-[3-morpholine-4-ylphenyl)amino]ethanol, 3-amino-4-(2-methoxyethoxy)-5-methylphenyl amine, 1-amino-3-bis-(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazole-5-on, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline, or mixtures of these compounds or the physiologically compatible salts thereof.

In a preferred embodiment, compositions (B) used according to the invention comprise one or more oxidation dye precursors in a total amount of 0.01 to 30.0 wt. %, preferably of 0.1 to 15 wt. %, more preferably of 0.6 to 3.1 wt. %, and especially particularly preferably of 1.2 to 2.2 wt. %, based on the weight of composition (B) used according to the invention.

In a further preferred embodiment, the oxidative treatment agents according to the invention, or compositions (B) used according to the invention, additionally comprise at least one further direct dye. Direct dyes can be broken down into anionic, cationic and non-ionic direct dyes. The direct dyes are preferably selected from the nitrophenylenediamines, the nitroaminophenols, the azo dyes, the anthraquinones, the triarylmethane dyes or the indophenols, and the physiologically compatible salts. The direct dyes are each preferably present in a total amount of 0.001 to 2 wt. %, based on the weight of composition (B) used according to the invention, or based on the weight of the oxidative treatment agent according to the invention. Direct dyes are used in oxidative coloring agents to nuance the hue that is achieved, and in oxidative blonding agents to compensate for undesirable red hues that may be created during the depletion of melanin naturally present in hair.

Preferred anionic direct dyes are the compounds known under the international designations or trade names Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1, Acid Black 52, bromophenol blue and tetrabromophenol blue.

Preferred cationic direct dyes are cationic triphenylmethane dyes, such as Basic Blue 7, Basic Blue 26, Basic Violet 2 and Basic Violet 14, aromatic systems substituted with a quaternary nitrogen group, such as Basic Yellow 57, Basic Red 76, Basic Blue 99, Basic Brown 16 and Basic Brown 17, cationic anthraquinone dyes such as HC Blue 16 (Bluequat B), and direct dyes that contain a heterocycle having at least one quaternary nitrogen atom, in particular Basic Yellow 87, Basic Orange 31 and Basic Red 51. The cationic direct dyes that are sold under the trademark Arianor are cationic direct dyes that are likewise preferred according to the invention.

In particular, nonionic nitro and quinone dyes and neutral azo dyes are suited as nonionic direct dyes. Preferred nonionic direct dyes are the compounds known under the international designations or trade names HC Yellow 2, HC Yellow 4, HC Yellow 5, HC Yellow 6, HC Yellow 12, HC Orange 1, Disperse Orange 3, HC Red 1, HC Red 3, HC Red 7, HC Red 10, HC Red 11, HC Red 13, HC Red BN, HC Blue 2, HC Blue 11, HC Blue 12, Disperse Blue 3, HC Violet 11, Disperse Violet 1, Disperse Violet 4, Disperse Black 9, as well as 1,4-diamino-2-nitrobenzene, 2-amino-4-nitrophenol, 1,4-bis-(2-hydroxyethyl)-amino-2-nitrobenzene, 3-nitro-4-(2-hydroxyethyl)-aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 4-[(3-hydroxypropyl)amino]-3-nitrophenol, 4-nitro-o-phenylenediamine, 6-nitro-1,2,3,4-tetrahydroquinoxaline, 2-hydroxy-1,4-naphthoquinone, picramic acid and the salts thereof, 2-amino-6-chloro-4-nitrophenol, 4-ethylamino-3-nitrobenzoic acid and 2-chloro-6-ethylamino-4-nitrophenol.

Preferred oxidative treatment agents according to the invention and/or compositions (B) preferably used according to the invention comprise at least one permanently cationic polymer B as a further optional ingredient.

In addition to at least one permanently cationically charged monomer type, the permanent cationic polymer preferably also comprises at least one permanently anionically charged monomer type, wherein the cationic monomers are present in a molar excess in relation to the anionic monomers so that the at least one second polymer according to the invention has a cationic net charge. Such preferred polymers according to the invention are also referred to as amphoteric or zwitterionic polymers.

In a first preferred embodiment, preferred oxidative treatment agents according to the invention and/or compositions (B) preferably used according to the invention comprise at least one permanently cationic polymer, which is selected from
cationic polymers composed of monomers with quaternary ammonium groups of the general formula (IIa)

R³—CH═CR⁴—CO—Z—(CₙH₂ₙ)—N⁽⁺⁾R⁵R⁶R⁷A⁽⁻⁾ (IIa),
in which R³ and R⁴, independently of one another, denote hydrogen or a methyl group, R⁵, R⁶, and R⁷, independently of one another, denote an alkyl group having 1 to 4 carbon atoms, Z denotes an NH group or an oxygen atom, n denotes an integer from 2 to 4, and A⁽⁻⁾ represents the anion of an inorganic or organic acid,
   preferably selected from cationic polymers composed of acrylamidopropyltrimethylammonium chloride,
   particularly preferably selected from amphoteric polymers having a cationic net charged, which by way of polymerization are composed of
a) cationic monomers with quaternary ammonium groups of general formula (IIa),

R³—CH═CR⁴—CO—Z—(CₙH₂ₙ)—N⁽⁺⁾R⁵R⁶R⁷A⁽⁻⁾ (IIa),
in which R³ and R⁴, independently of one another, denote hydrogen or a methyl group, R⁵, R⁶, and R⁷, independently of one another, denote an alkyl group having 1 to 4 carbon atoms, Z denotes an NH group or an oxygen atom, n denotes an integer from 2 to 4, and A⁽⁻⁾ represents the anion or an inorganic or organic acid, and
b) at least one unsaturated carboxylic acid, selected from acrylic acid, methacrylic acid, and crotonic acid, and mixtures of these acids, wherein the at least one unsaturated carboxylic acid may be present in the form of the salts thereof,
   wherein the cationic monomers are present in the polymer in a molar excess in relation to the anionic monomers,
   exceptionally preferably selected from amphoteric polymers having a cationic net charge, which comprise the at least one monomer type of the general formula (IIa) and the at least one monomer type of the unsaturated carboxylic acid, selected from acrylic acid, methacrylic acid and crotonic acid, and mixtures thereof, in a molar ratio of 60:40 to 95:5, and preferably of 75:25 to 90:10 in relation to each other,
   exceptionally preferably selected from amphoteric copolymers having a cationic net charge, which are composed of acrylamidopropyltrimethylammonium chloride and acrylic acid in a molar ratio of 60:40 to 95:5, and preferably of 75:25 to 90:10 in relation to each other;
   cellulose 2-[2-hydroxy-3-(trimethylammonio)propoxy]ethyl ether chloride, which is available, for example, under the INCI name Polyquaternium-10,
   terpolymers of acrylic acid, diallyldimethylammonium chloride and acrylamide, such as are available, for example, under the INCI name Polyquaternium-39,
   homopolymers of N,N,N-trimethyl-2-[(methyl-1-oxo-2-propenyl)oxy]ethanaminium chloride, such as are available, for example, under the INCI name Polyquaternium-37,
   copolymers of diallyldimethylammonium chloride and acrylic acid, such as are available, for example, under the INCI name Polyquaternium-22,
   hydroxyethyl cellulose/diallyldimethylammonium chloride copolymers, such as are available, for example, under the INCI name Polyquaternium-4,
   copolymers of acrylamide and beta-methacrylyloxyethyl trimethyl ammonium methosulfate, such as are available, for example, under the INCI name Polyquaternium-5,
   homopolymers of N,N-dimethyl-N-2-propenyl-2-propene-1-aminium chloride, such as are available, for example, under the INCI name Polyquaternium-6,
   copolymers of diallyldimethylammonium chloride and acrylamide, such as are available, for example, under the INCI name Polyquaternium-7,
   copolymers of vinylpyrrolidone and dimethylaminoethyl methacrylate diethyl sulfate, such as are available, for example, under the INCI name Polyquaternium-11,
   and mixtures of the aforementioned polymers.

Exceptionally preferred permanently cationic polymers according to the invention are selected from cellulose 2-[2-hydroxy-3-(trimethylammonio)propoxy]ethyl ether chloride, amphoteric copolymers having a cationic net charge that are composed of acrylamidopropyltrimethylammonium chloride and acrylic acid in a molar ratio of 60:40 to 95:5, and preferably of 75:25 to 90:10 in relation to each other, and terpolymers of acrylic acid, diallyldimethylammonium chloride and acrylamide, and mixtures of two and three of these polymers.

Particularly preferred polymer B mixtures comprise cellulose 2-[2-hydroxy-3-(trimethylammonio)propoxy]ethyl ether chloride and at least one amphoteric copolymer having a cationic net charge that is composed of acrylamidopropyltrimethylammonium chloride and acrylic acid in a molar ratio of 60:40 to 95:5, and preferably of 75:25 to 90:10 in relation to each other.

Further particularly preferred polymer B mixtures comprise cellulose 2-[2-hydroxy-3-(trimethylammonio)propoxy]ethyl ether chloride, at least one amphoteric copolymer having a cationic net charge that is composed of acrylamidopropyltrimethylammonium chloride and acrylic acid in a molar ratio of 60:40 to 95:5, and preferably of 75:25 to 90:10 in relation to each other, and at least one terpolymer of acrylic acid, diallyldimethylammonium chloride and acrylamide.

Likewise, exceptionally preferred permanently cationic polymers B according to the invention are selected from Polyquaternium-10, amphoteric copolymers having a cationic net charge that are composed of acrylamidopropyltrimethylammonium chloride and acrylic acid in a molar ratio of 60:40 to 95:5, and preferably of 75:25 to 90:10 in relation to each other, and Polyquaternium-39, and mixtures of two and three of these polymers.

Further particularly preferred polymer B mixtures comprise Polyquaternium-10 and at least one amphoteric copolymer having a cationic net charge that is composed of acrylamidopropyltrimethylammonium chloride and acrylic acid in a molar ratio of 60:40 to 95:5, and preferably of 75:25 to 90:10 in relation to each other.

Further particularly preferred polymer B mixtures comprise Polyquaternium-10 and at least one amphoteric copolymer having a cationic net charge that is composed of acrylamidopropyltrimethylammonium chloride and acrylic acid in a molar ratio of 60:40 to 95:5, and preferably of 75:25 to 90:10 in relation to each other, and Polyquaternium-39.

Preferred oxidative treatment agents according to the invention comprise the at least one permanently cationic polymer B in a total amount of 0.05 to 1.5 wt. %, preferably 0.1 to 1.0 wt. %, and particularly preferably 0.2 to 0.8 wt. %, in each case based on the weight of the oxidative treatment agent according to the invention.

Preferred compositions (B) used according to the invention include the at least one permanently cationic polymer B in a total amount of 0.05 to 1.5 wt. %, preferably 0.1 to 1.0 wt. %, and particularly preferably 0.2 to 0.8 wt. %, in each case based on the weight of composition (B) used according to the invention.

Further preferred treatment agents according to the invention and/or compositions (B) preferably used according to the invention comprise at least one permanently cationic polymer B, selected from
cationic polymers composed of monomers with quaternary ammonium groups of the general formula (IIa)

R³—CH═CR⁴—CO—Z—(CₙH₂ₙ)—N⁽⁺⁾R⁵R⁶R⁷A⁽⁻⁾ (IIa),
in which R³ and R⁴, independently of one another, denote hydrogen or a methyl group, R⁵, R⁶, and R⁷, independently of one another, denote an alkyl group having 1 to 4 carbon atoms, Z denotes an NH group or an oxygen atom, n denotes an integer from 2 to 4, and A″ represents the anion of an inorganic or organic acid,
preferably selected from cationic polymers composed of acrylamidopropyltrimethylammonium chloride,
particularly preferably selected from amphoteric polymers having a cationic net charged, which by way of polymerization are composed of
c) cationic monomers with quaternary ammonium groups of general formula (IIa),

R³—CH═CR⁴—CO—Z—(CₙH₂ₙ)—N⁽⁺⁾R⁵R⁶R⁷A⁽⁻⁾ (IIa),
in which R³ and R⁴, independently of one another, denote hydrogen or a methyl group, R⁵, R⁶, and R⁷, independently of one another, denote an alkyl group having 1 to 4 carbon atoms, Z denotes an NH group or an oxygen atom, n denotes an integer from 2 to 4, and A⁽⁻⁾ represents the anion or an inorganic or organic acid, and
d) at least one unsaturated carboxylic acid, selected from acrylic acid, methacrylic acid, and crotonic acid, and mixtures of these acids, wherein the at least one unsaturated carboxylic acid may be present in the form of the salts thereof,
wherein the collectivity of all cationic monomers is present in the polymer in a molar excess in relation to the collectivity of all anionic monomers;
exceptionally preferably selected from amphoteric polymers having a cationic net charge, which comprise the at least one monomer type of the general formula (IIa) and the at least one monomer type of the unsaturated carboxylic acid, selected from acrylic acid, methacrylic acid and crotonic acid, and mixtures thereof, in a molar ratio of 60:40 to 95:5, and preferably of 75:25 to 90:10 in relation to each other,
exceptionally preferably selected from amphoteric copolymers having a cationic net charge, which are composed of acrylamidopropyltrimethylammonium chloride and acrylic acid in a molar ratio of 60:40 to 95:5, and preferably of 75:25 to 90:10 in relation to each other;
cellulose 2-[2-hydroxy-3-(trimethylammonio)propoxy] ethyl ether chloride, which is available, for example, under the INCI name Polyquaternium-10,
terpolymers of acrylic acid, diallyldimethylammonium chloride and acrylamide, such as are available, for example, under the INCI name Polyquaternium-39,
homopolymers of N,N,N-trimethyl-2-[(methyl-1-oxo-2-propenyl)oxy]ethanaminium chloride, such as are available, for example, under the INCI name Polyquaternium-37,
copolymers of diallyldimethylammonium chloride and acrylic acid, such as are available, for example, under the INCI name Polyquaternium-22,
hydroxyethyl cellulose/diallyldimethylammonium chloride copolymers, such as are available, for example, under the INCI name Polyquaternium-4,
copolymers of acrylamide and beta-methacrylyloxyethyl trimethyl ammonium methosulfate, such as are available, for example, under the INCI name Polyquaternium-5,
homopolymers of N,N-dimethyl-N-2-propenyl-2-propene-1-aminium chloride, such as are available, for example, under the INCI name Polyquaternium-6,
copolymers of diallyldimethylammonium chloride and acrylamide, such as are available, for example, under the INCI name Polyquaternium-7, and
copolymers of vinylpyrrolidone and dimethylaminoethyl methacrylate diethyl sulfate, such as are available, for example, under the INCI name Polyquaternium-11,
   in a total amount of 0.05 to 1.5 wt. %, preferably 0.1 to 1.0 wt. %, and particularly preferably 0.2 to 0.8 wt. %, in each case based on the weight of the oxidative treatment agent according to the invention or the weight of composition (B) used according to the invention.
   Further preferred oxidative treatment agents according to the invention and/or compositions (B) preferably used according to the invention comprise cellulose 2-[2-hydroxy-3-(trimethylammonio)propoxy] ethyl ether chloride and at least one amphoteric copolymer having a cationic net charge that is composed of acrylamidopropyltrimethylammonium chloride and acrylic acid in a molar ratio of 60:40 to 95:5, and preferably of 75:25 to 90:10 in relation to each other, in a total amount of 0.05 to 1.5 wt. %, preferably 0.1 to 1.0 wt. %, and particularly preferably 0.2 to 0.8 wt. %, in each case based on the weight of the oxidative treatment agent according to the invention or the weight of composition (B) used according to the invention.

Further preferred oxidative treatment agents according to the invention and/or compositions (B) preferably used according to the invention comprise cellulose 2-[2-hydroxy-3-(trimethylammonio)propoxy] ethyl ether chloride, at least one amphoteric copolymer having a cationic net charge that is composed of acrylamidopropyltrimethylammonium chloride and acrylic acid in a molar ratio of 60:40 to 95:5, and preferably of 75:25 to 90:10 in relation to each other, and at least one terpolymer of acrylic acid, diallyldimethylammonium chloride and acrylamide in a total amount of 0.05 to 1.5 wt. %, preferably 0.1 to 1.0 wt. %, and particularly preferably 0.2 to 0.8 wt. %, in each case based on the weight of the oxidative treatment agent according to the invention or the weight of composition (B) used according to the invention.

Further particularly preferred oxidative treatment agents and/or compositions (B) preferably used according to the invention comprise Polyquaternium-10 and at least one amphoteric copolymer having a cationic net charge that is composed of acrylamidopropyltrimethylammonium chloride and acrylic acid in a molar ratio of 60:40 to 95:5, and preferably of 75:25 to 90:10 in relation to each other, in a total amount of 0.05 to 1.5 wt. %, preferably 0.1 to 1.0 wt. %, and particularly preferably 0.2 to 0.8 wt. %, in each case based on the weight of the oxidative treatment agent according to the invention or the weight of composition (B) used according to the invention.

Further particularly preferred oxidative treatment agents and/or compositions (B) preferably used according to the invention comprise Polyquaternium-10 and at least one amphoteric copolymer having a cationic net charge that is composed of acrylamidopropyltrimethylammonium chloride and acrylic acid in a molar ratio of 60:40 to 95:5, and preferably of 75:25 to 90:10 in relation to each other, and Polyquaternium-39 in a total amount of 0.05 to 1.5 wt. %, preferably 0.1 to 1.0 wt. %, and particularly preferably 0.2 to 0.8 wt. %, in each case based on the weight of the oxidative treatment agent according to the invention or the weight of composition (B) used according to the invention.

A further subject matter of the present invention is a method for oxidatively coloring and/or lightening keratin fibers, and in particular human hair, in which an oxidative treatment agent is applied to the keratin fibers, and in particular to the human hair, and optionally after an exposure time of 0.1 to 60 minutes, preferably 1 to 50 minutes, particularly preferably 10 to 45 minutes, and exceptionally preferably 30 to 45 minutes, is rinsed off again, wherein this treatment agent comprises
b) at least one dicarboxylic acid having 2 to 10 carbon atoms and/or at least one salt of this acid/these acids,
c) at least one amino acid,
d) hydrogen peroxide, and
e) Water
   wherein the oxidative treatment agent has a pH value in the range of 2.5 to 6.5, preferably in the range of 3 to 5.5, and particularly preferably in the range of 3.5 to 5.0, each measured at 20° C.

What was said above with respect to the oxidative treatment agents according to the invention and preferred oxidative treatment agents according to the present invention applies, mutatis mutandis, to the method according to the invention for oxidatively coloring and/or lightening keratin fibers, and in particular human hair, and the preferred embodiments thereof.

The method according to the invention for oxidatively coloring and/or lightening keratin fibers, and in particular human hair, may optionally be followed by further hair treatment steps, such as the application of a conditioner, a hair styling agent, such as a straightening agent or a perming agent, a further hair coloring agent, for example for coloring or blonding highlights, rinsing steps, and drying steps, for example for rubbing dry or pressing dry by way of a towel, blow drying, or drying by way of a hood dryer.

Mixing Ratios of Oxidative Treatment Agents According to the Invention and Composition (B)

It has proven useful according to the invention if the weight ratio of the oxidative treatment agent according to the invention, which comprises water, hydrogen peroxide, and the hair-protecting combination of at least one dicarboxylic acid having 2 to 10 carbon atoms and at least one amino acid, and preferably at least one amino acid of formula (IV), and has a pH value in the range of 2.5 to 6.5, preferably in the range of 3 to 5.5, and particularly preferably in the range of 3.5 to 5.0, each measured at 20° C., to composition (B), comprising at least one alkalizing agent, selected from ammonium hydroxide, monoethanolamine and sodium silicates, and mixtures thereof, optionally water, and optionally at least one oxidation dye precursor and/or at least one direct dye, is in the range of 10:1 to 1:4, preferably 5:1 to 1:3, particularly preferably 4:1 to 1:2, and exceptionally preferably 2:1 to 1:1.

What was said above with respect to the oxidative treatment agents according to the invention and preferred oxidative treatment agents according to the present invention applies, mutatis mutandis, to the method according to the invention for oxidatively coloring and/or lightening keratin fibers, and in particular human hair, using the at least two above-described compositions (A) and (B), and the preferred embodiments thereof.

### EXAMPLES

The following examples of oxidative treatment agents according to the invention are intended to illustrate the invention, without thereby limiting the invention to these examples.

TABLE 1
Oxidative treatment agents (developers) for oxidative hair dyes
and blonding agents (all quantity information is in % by weight)

| Ingredient | No. 1 | No. 2 | No. 3 | No. 4 | No. 5 |
|---|---|---|---|---|---|
| Cetearyl alcohol | 2.8 | 2.2 | 5 | 5 | 5 |
| PEG-40 castor oil | 0.6 | 0.4 | 1.5 | 1.5 | 1.5 |
| Sodium cetearyl sulfate | 0.4 | 0.3 | 1.0 | 1.0 | 1.0 |
| Paraffin oil | 21.0 | 13.0 | 31.0 | — | — |
| Isopropyl myristate | — | — | — | 15.0 | 31.0 |
| Etidronic acid | 0.15 | 0.15 | 0.15 | 0.15 | 0.15 |
| L-Arginine | 0.4 | 0.4 | — | 0.4 | — |
| L-Lysine hydrochloride | — | — | 0.7 | — | 0.7 |
| Succinic acid | 1.0 | 1.0 | 1.0 | 1.0 | 1.0 |
| Disodium | 0.1 | 0.1 | 0.1 | 0.1 | 0.1 |
| pyrophosphate | | | | | |
| Dipicolinic acid | 0.1 | 0.1 | 0.1 | 0.1 | 0.1 |
| Sodium benzoate | 0.04 | 0.04 | 0.04 | 0.04 | 0.04 |
| Potassium hydroxide | 0.12 | 0.12 | 0.12 | 0.12 | 0.12 |
| Hydrogen peroxide | 6.0 | 9.0 | 12.0 | 9.0 | 12.0 |
| Water (demineralized) | to make | to make | to make | to make | to make |
| | 100 | 100 | 100 | 100 | 100 |

TABLE 2
Oxidative treatment agents (developers) for oxidative hair dyes
and blonding agents (all quantity information is in % by weight)

| Ingredient | No. 1 | No. 2 | No. 3 | No. 4 | No. 5 |
|---|---|---|---|---|---|
| Cetearyl alcohol | 2.8 | 2.2 | 5 | 5 | 5 |
| PEG-40 castor oil | 0.6 | 0.4 | 1.5 | — | 1.5 |
| Sodium cetearyl sulfate | 0.4 | 0.3 | 1.0 | — | — |
| Paraffin oil | 21.0 | 13.0 | 31.0 | — | — |
| Isopropyl myristate | — | — | — | 15.0 | 31.0 |
| Trideceth-2 | — | — | — | 2.0 | 2.0 |
| carboxamide MEA | | | | | |
| Etidronic acid | 0.15 | 0.15 | 0.15 | 0.15 | 0.15 |
| L-Arginine | 0.4 | 0.4 | — | 0.4 | — |
| L-Lysine hydrochloride | — | — | 0.7 | — | 0.7 |
| Maleic acid | 1.0 | 1.0 | — | 1.0 | 1.0 |
| Fumaric acid | — | — | 1.0 | — | — |
| Disodium | 0.1 | 0.1 | 0.1 | 0.1 | 0.1 |
| pyrophosphate | | | | | |
| Dipicolinic acid | 0.1 | 0.1 | 0.1 | 0.1 | 0.1 |
| Sodium benzoate | 0.04 | 0.04 | 0.04 | 0.04 | 0.04 |
| Potassium hydroxide | 0.12 | 0.12 | 0.12 | 0.12 | 0.12 |
| Hydrogen peroxide | 6.0 | 9.0 | 12.0 | 9.0 | 12.0 |
| Water (demineralized) | to make | to make | to make | to make | to make |
| | 100 | 100 | 100 | 100 | 100 |

TABLE 3
Oxidative treatment agents (developers) for oxidative hair dyes
and blonding agents (all quantity information is in % by weight)

| Ingredient | No. 1 | No. 2 | No. 3 | No. 4 | No. 5 |
|---|---|---|---|---|---|
| 1,2-propylene glycol | 2.0 | 2.0 | 2.0 | 2.0 | 2.0 |
| Cetearyl alcohol | 1.5 | 1.5 | 3.6 | 1.5 | 3.6 |
| Ceteareth-20 | 0.6 | 0.4 | 1.5 | 1.5 | 1.5 |
| Trideceth-2 | — | — | 2.0 | — | 2.0 |
| carboxamide MEA | | | | | |
| Paraffin oil | 2.0 | 13.0 | 31.0 | — | — |
| Isopropyl myristate | — | — | — | 2.0 | 31.0 |
| Etidronic acid | 0.15 | 0.15 | 0.15 | 0.15 | 0.15 |
| L-Arginine | 0.4 | 0.4 | — | 0.4 | — |
| L-Lysine hydrochloride | — | — | 0.7 | — | 0.7 |
| Disodium succinate | 1.5 | 2.0 | 2.0 | 2.0 | 1.5 |
| hexahydrate | | | | | |
| Steardimonium | 0.1 | 0.1 | 0.1 | 0.1 | 0.1 |
| hydroxypropyl | | | | | |
| hydrolyzed keratin | | | | | |
| Disodium | 0.1 | 0.1 | 0.1 | 0.1 | 0.1 |
| pyrophosphate | | | | | |
| Dipicolinic acid | 0.1 | 0.1 | 0.1 | 0.1 | 0.1 |
| Sodium benzoate | 0.04 | 0.04 | 0.04 | 0.04 | 0.04 |
| Potassium hydroxide | 0.2 | 0.2 | 0.2 | 0.2 | 0.2 |
| Hydrogen peroxide | 6.0 | 9.0 | 12.0 | 6.0 | 9.0 |
| Water (demineralized) | to make | to make | to make | to make | to make |
| | 100 | 100 | 100 | 100 | 100 |

TABLE 4
Coloring creams (all quantity information is in % by weight)

| Formulation components (color créme) | 1 | 2 |
|---|---|---|
| Cetyl alcohol | 3.6 | 4.6 |
| Behenyl alcohol | 2.4 | 3.0 |
| Paraffinum Liquidum | 2.1 | 2.1 |
| Eumulgin B 3 (INCI: Ceteareth-30) | 1.2 | 1.8 |
| Brij S 100 PA (stearyl alcohol ethoxylated (100 EO)) | 0.6 | 1.0 |
| Cutina GMS (INCI: Glyceryl Stearate) | 0.6 | 0.6 |
| Propylene glycol | 6.0 | 6.0 |
| p-toluylenediamine sulfate | 1.50 | 1.50 |
| Resorcinol | 0.58 | 0.58 |
| m-aminophenol | 0.16 | 0.16 |
| 3-amino-2-methylamino-6-methoxypyridine | 0.05 | 0.05 |
| Potassium hydroxide (50%) | 0.7 | 0.7 |
| Ethylenediaminetetraacetic acid, tetrasodium | 0.20 | 0.20 |
| salt | | |
| Sodium sulfite (anhydrous) | 0.30 | 0.30 |
| Vitamin C | 0.05 | 0.05 |
| Product W 37194 (N,N,N-trimethyl-3-[(1-oxo- | 3.75 | 3.0 |
| 2-propenyl)amino]-1-propanaminium | | |
| chloride, polymer with sodium 2-propenoate) | | |
| (INCI: Acrylamidopropyltrimonium chloride/ | | |
| Acrylate Copolymer), 20 wt. % aqueous | | |
| solution | | |
| Ammonia (25 wt. % aqueous solution) | 5.80 | 5.80 |
| Perfume | 0.40 | 0.40 |
| Water | to make | to make |
| | 100 | 100 |

Color crémes 1 and 2 according to Table 4 correspond to compositions (B) provided in the description. They were each mixed with one of the oxidative treatment agents from Tables 1, 2 or 3 in a weight ratio of 1:1, applied directly thereafter to human hair strands, and rinsed off with water and a shampoo after an exposure time of 45 minutes. Afterwards, the hair was first dried with a hand towel and then with a hair dryer.

TABLE 5
Blonding powder (powdery composition (B))

| Formulation components | (% by weight) |
|---|---|
| Sodium silicate (SiO₂:Na₂O = 2:1) | 36.00 |
| Magnesium carbonate | 12.85 |
| Sodium hexametaphosphate | 0.20 |
| Rohagit S hv (methylmethacrylate/methacrylic acid | 1.00 |
| copolymer) | |
| EDTA disodium salt (ethyleneediaminetetraacetic acid, | 0.60 |
| disodium salt) | |
| Polyquaternium-4 | 0.30 |
| Silicic acid, hydrophilic | 0.40 |
| Glycine | 0.60 |
| Potassium persulfate | 32.00 |
| Ammonium persulfate | 10.00 |
| CI 77007 (ULTRAMARINES) | 0.15 |
| Dimethicone (85 wt. %), dimethiconol (15 wt. %) | 1.50 |
| Paraffinum Liquidum | 3.80 |

To produce the ready-to-use blonding agent, 2 parts by weight of an oxidative treatment agent from Tables 1, 2 or 3 were mixed with one part by weight of the blonding powder according to Table 5, applied directly thereafter to human hair strands, and rinsed off with water and a shampoo after an exposure time of 50 minutes. Afterwards, the hair was first dried with a hand towel and then with a hair dryer.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An oxidative treatment agent for keratin fibers, in particular for human hair, comprising:
a) at least one dicarboxylic acid having 2 to 10 carbon atoms and/or at least one salt of this acid/these acids,
b) at least one amino acid,
c) hydrogen peroxide in an amount of 0.5 to 13 wt. %, when calculated as 100% H₂O₂ and based on the total weight of the oxidative treatment agent, and
d) water in an amount of 20 to 95 wt. % based on the total weight of the oxidative treatment agent according to the invention,
wherein the oxidative treatment agent has a pH value in the range of 2.5 to 6.5 measured at 20° C.

2. The treatment agent according to claim 1, wherein the at least one amino acid is selected from amino acids of formula (VI) 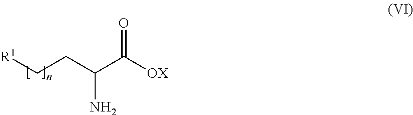
wherein
X denotes a hydrogen atom or a monovalent or divalent cation;
n denotes zero, 1, 2 or 3; and
R¹ denotes a group selected from an amino group, a guanidine group, a (1H-imidazole-4-yl) group, a carboxylic acid amid group —CONH₂, a 1H-indole-3-yl group, a thiol group —SH, and a methylsulfanyl group —SCH₃, or at least one salt of these amino acids.

3. The treatment agent according to claim 1, wherein the at least one dicarboxylic acid having 2 to 10 carbon atoms is selected from the group consisting of maleic acid, fumaric acid, succinic acid, malic acid, oxalic acid, malonic acid, adipic acid, pimelic acid, suberic acid, azelaic acid, sebacic acid, D-tartaric acid, L-tartaric acid, mesotartaric acid, racemic tartaric acid, alpha-ketoglutaric acid, beta-ketoglutaric acid and mixtures of these acids.

4. The treatment agent according to claim 1, wherein the at least one dicarboxylic acid having 2 to 10 carbon atoms is succinic acid.

5. The treatment agent according to claim 1, wherein the at least one dicarboxylic acid having 2 to 10 carbon atoms is present in a total amount of 0.1 to 5 wt. %, each converted to the undissociated acid and based on the weight of the treatment agent.

6. The treatment agent according to claim 2, wherein the at least one amino acid of formula (VI) is selected from the group consisting of arginine, lysine, histidine, asparagine, glutamine, cysteine, methionine, tryptophan, and mixtures thereof.

7. The treatment agent according to claim 6, wherein the at least one amino acid of formula (VI) includes a mixture of arginine and lysine

8. The treatment agent according to claim 1, wherein the at least one amino acid is present in a total amount of 0.05 to 3 wt. %, each converted to the undissociated acid and based on the weight of the treatment agent.

9. The treatment agent according to claim 1, further including at least one chelating agent in a total amount of 0.01 to 2.5 wt. % based on the weight of the oxidative treatment agent.

10. The treatment agent according to claim 1, further including at least one compound of the general formula (III), 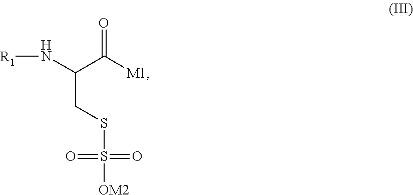
wherein
R1 denotes a hydrogen atom or a structural element of formula (IV) 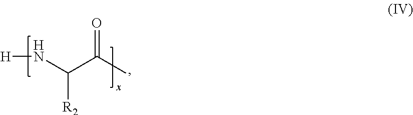
wherein
a denotes an integer from 1 to 100,
the group R2 in each of the structural elements of formula (IV) is selected independently of the preceding structural element of formula (IV),
R2 denotes a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazole-4-ylmethyl group, a 1H-indole-3-ylmethyl group or a (sulfosulfanyl)methyl group,
M1 denotes the group —OM2 or a structural element of formula (V), 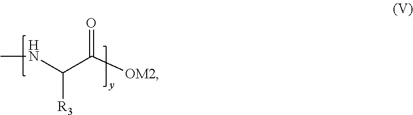
wherein
y denotes an integer from 1 to 100,
the group R3 in each of the structural elements of formula (V) is selected independently of the preceding structural element of formula (V),
R3 denotes a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazole-4-ylmethyl group, a 1H-indole-3-ylmethyl group or a (sulfosulfanyl)methyl group,
M2 denotes a hydrogen atom, an equivalent of a monovalent or polyvalent cation, or an ammonium ion (NH₄)⁺,
wherein preferably one or more compounds of the above-described formula (III) is present in a total amount of 0.001 to 2.5 wt. % based on the weight of the treatment agent.

11. The treatment agent according to claim 1, further including at least one fatty acid amide of formula (AMID-I) in a total amount of 0.1 to 10 wt. % based on the weight of the treatment agent, 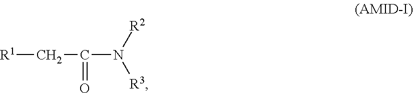
wherein
R¹ is selected from linear or branched C4 to C22 alkyl groups, which are saturated or unsaturated, wherein the alkyl and alkenyl groups are unsubstituted or may be substituted with oxygen atoms, nitrogen atoms, hydroxyl groups, ether groups, oxyalkylene groups, polyoxyalkylene groups, carboxylic acid groups, amine groups, amide groups, halogen-containing groups, ester groups, siloxane groups, or polysiloxane groups,
and alkoxylated alkyl groups of formulas: 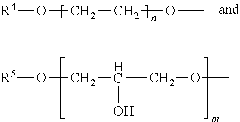
wherein:
R⁴ and R⁵, which can be the same or different, are selected from linear and branched C4 to C22 alkyl groups, which are saturated or unsaturated,
n is an integer in the range from 1 to 10, and preferably in the range from 1 to 4; and
m is an integer in the range from 1 to 6; and
R² and R³, which may be the same or different, are each independently of one another selected from H; linear or branched C1 to C22 alkyl groups, which are saturated or unsaturated, wherein the alkyl and alkenyl groups are unsubstituted or substituted with oxygen atoms, nitrogen atoms, hydroxyl groups, ether groups, oxyalkylene groups, and in particular oxyethylene groups, polyoxyalkylene groups, and in particular polyoxyethylene groups, carboxylic acid groups, amine groups, alkylamine groups, and in particular C1 to C3 alkylamine groups, dialkylamine groups, and in particular di-(C1-C3)-alkylamine groups, substituted and non-substituted 4,5-dihydroimidazole groups, amide groups, halogen-containing groups, ester groups, siloxane groups, or polysiloxane groups.

12. A method for oxidatively coloring and/or lightening keratin fibers, and in particular human hair, wherein a treatment agent according to claim 1 is applied to the keratin fibers, and in particular to the human hair, and optionally is rinsed off again after an exposure time of 0.1 to 60 minutes.

13. A method for oxidatively coloring and/or lightening keratin fibers, and in particular human hair, comprising:
providing an oxidative treatment agent (A), comprising
a) at least one dicarboxylic acid having 2 to 10 carbon atoms and/or at least one salt of this acid/these acids, and which is selected from the group consisting of maleic acid, fumaric acid, succinic acid, malic acid, oxalic acid, malonic acid, adipic acid, pimelic acid, suberic acid, azelaic acid, sebacic acid, D-tartaric acid, L-tartaric acid, mesotartaric acid, racemic tartaric acid, alpha-ketoglutaric acid, beta-ketoglutaric acid, and mixtures of these acids,
b) at least one amino acid, selected from amino acids of formula (VI) 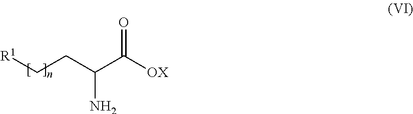
wherein
X denotes a hydrogen atom or a monovalent or divalent cation;
n denotes zero, 1, 2 or 3; and
R¹ denotes a group selected from an amino group, a guanidine group, a (1H-imidazole-4-yl) group, a carboxylic acid amid group —CONH₂, a 1H-indole-3-yl group, a thiol group —SH, and a methylsulfanyl group —SCH₃, or at least one salt of these amino acids,
c) hydrogen peroxide,
d) water,
e) optionally at least one substance selected from
compounds of the general formula (III), 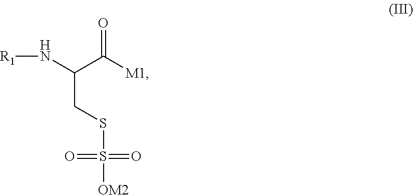
wherein
R1 denotes a hydrogen atom or a structural element of formula (IV) 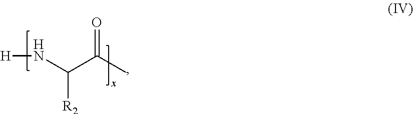
wherein
a denotes an integer from 1 to 100,
the group R2 in each of the structural elements of formula (IV) can each be selected independently of the preceding structural element of formula (IV),
R2 denotes a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazole-4-ylmethyl group, a 1H-indole-3-ylmethyl group or a (sulfosulfanyl)methyl group,
M1 denotes the group —OM2 or a structural element of formula (V), 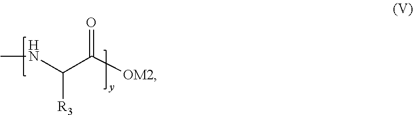
wherein
y denotes an integer from 1 to 100,
the group R3 in each of the structural elements of formula (V) can each be selected independently of the preceding structural element of formula (V),
R3 denotes a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazole-4-ylmethyl group, a 1H-indole-3-ylmethyl group or a (sulfosulfanyl)methyl group,
M2 denotes a hydrogen atom, an equivalent of a monovalent or polyvalent cation, or an ammonium ion (NH₄)⁺,
at least one chelating agent in a total amount of 0.01 to 2.5 wt. % based on the weight of the oxidative treatment agent (A), and
at least one fatty acid amide of formula (AMID-I) in a total amount of 0.1 to 10 wt. % based on the weight of the oxidative treatment agent (A), 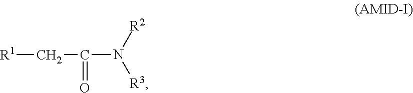
wherein
R¹ is selected from linear and branched C4 to C22 alkyl groups, which are saturated or unsaturated, wherein the alkyl and alkenyl groups are unsubstituted or may be substituted with oxygen atoms, nitrogen atoms, hydroxyl groups, ether groups, oxyalkylene groups, polyoxyalkylene groups, carboxylic acid groups, amine groups, amide groups, halogen-containing groups, ester groups, siloxane groups, or polysiloxane groups,
and alkoxylated alkyl groups of formulas: 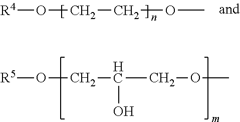
wherein:
R⁴ and R⁵, which can be the same or different, are selected from linear or branched C4 to C22 alkyl groups, which are saturated or unsaturated,
n is an integer in the range from 1 to 10; and
m is an integer in the range from 1 to 6; and
R² and R³, which may be the same or different, are each independently of one another selected from H; linear or branched C1 to C22 alkyl groups, which are saturated or unsaturated, wherein the alkyl and alkenyl groups are unsubstituted or substituted with oxygen atoms, nitrogen atoms, hydroxyl groups, ether groups, oxyalkylene groups, and in particular oxyethylene groups, polyoxyalkylene groups, and in particular polyoxyethylene groups, carboxylic acid groups, amine groups, alkylamine groups, and in particular C1 to C3 alkylamine groups, dialkylamine groups, and in particular di-(C1-C3)-alkylamine groups, substituted or non-substituted 4,5-dihydroimidazole groups, amide groups, halogen-containing groups, ester groups, siloxane groups, or polysiloxane groups,
wherein the oxidative treatment agent has a pH value in the range of 2.5 to 6.5 measured at 20° C.;
providing a composition (B), comprising
g) at least one alkalizing agent, selected from the group consisting of ammonium hydroxide, monoethanolamine and sodium silicates, and mixtures thereof,
h) optionally water, and
i) optionally at least one oxidation dye precursor and/or at least one direct dye, wherein composition (B) has a pH value in the range of 6.5 to 12.0 measured at 20° C.,
mixing compositions (A) and (B) with each other, wherein the weight ratio of (A) to (B) is in the range of 10:1 to 1:4, directly thereafter
applying the mixture of (A) and (B) to the keratin fibers, and in particular to the human hair,
rinsing after an exposure time of 0.1 to 60 minutes, and
optionally performing further heat treatments.

14. The method according to claim 13, wherein the oxidative treatment agent (A) comprises the at least one dicarboxylic acid having 2 to 10 carbon atoms in a total amount of 0.1 to 5 wt. %, each converted to the undissociated acid and based on the weight of the treatment agent (A).

15. The method according to claim 13, wherein the oxidative treatment agent (A) comprises the at least one amino acid in a total amount of 0.05 to 3 wt. %, each converted to the undissociated acid and based on the weight of the oxidative treatment agent (A).

16. The method according to any claim 13, wherein composition (B) is powdery and comprises at least one persulfate salt and/or at least one percarbonate salt.
